SEQUENCE LISTING (2 sequences)

SEQ ID NO: 1 - DNA, 2451 bp, Artificial Sequence
  cDNA encoding human p53
cgtgctttccacgacggtgacacgcttccctggattggccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcatgtttgtgcctgtcctgggagagaccggcgcacagaggaagagaatctccgcaagaaaggggagcctcaccacgagctgcccccagggagcactaagcgagcactgtccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttctttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccaaaaaaaaaaaaaaaaaaaa

SEQ ID NO: 2 - protein, 393 aa, Homo sapiens [sequence omitted]

(19) United States
(12) Patent Application Publication
(10) Pub. No.: US 2018/0106807 A1
(43) Pub. Date: Apr. 19, 2018

(54) DIAGNOSTIC AND PROGNOSTIC METHODS FOR BARRETT'S ESOPHAGUS

(71) Applicant: Miraca Life Sciences, Inc., Irving, TX (US)

(72) Inventor: Mark Redston, Needham, MA (US)

(21) Appl. No.: 15/788,699

(22) Filed: Oct. 19, 2017

Related U.S. Application Data
(60) Provisional application No. 62/410,038, filed on Oct. 19, 2016.

(51) Int. Cl. G01N 33/574; C12Q 1/68

(52) U.S. Cl. CPC G01N 33/57407; C12Q 1/6886; C12Q 2600/158; G01N 2800/50; G01N 2333/82

(57) ABSTRACT

Provided are methods, compositions, kits, and systems for determining the risk that a subject who does not have dysplasia will develop dysplasia and/or esophageal cancer.

### RELATED APPLICATION

This application claims the benefit of priority to U.S. Provisional Patent Application No. 62/410,038, filed Oct. 19, 2016, the entire content of which is incorporated herein by reference.

### INCORPORATION-BY-REFERENCE OF SEQUENCE LISTING

The contents of the text file named “43577-501001US_Sequence_Listing.txt”, which was created on Oct. 19, 2017 and is 6.81 KB in size, is hereby incorporated by reference in its entirety.

### FIELD OF THE INVENTION

The present invention relates to cancer diagnostics and therapeutics.

### BACKGROUND

Barrett's esophagus (BE) is the transformation of the normal squamous epithelium of the distal esophagus to a columnar intestinal-type epithelium. BE is thought to occur as a result of gastro-esophageal reflux. BE is the major precursor to esophageal cancer, and is associated with a 10-20 fold increased risk of progression to cancer. In patients with BE, esophageal cancer is typically preceded by dysplasia, and therefore patients are recommended to undergo routine endoscopic surveillance every 3-5 years in order to diagnose and treat dysplasia prior to the development of cancer. Progression remains relatively rare on an individual basis, and may occur despite screening. Therefore, biomarkers of progression risk are urgently needed to better manage this patient population.

### SUMMARY OF THE DISCLOSURE

The present subject matter provides, inter alia, a method for identifying whether a subject who does not have dysplasia is at risk of developing dysplasia or esophageal cancer. Related kits, compositions, and systems are also provided. The method may include, e.g., immunohistologically staining a biopsy from the subject for p53 expression, wherein said biopsy comprises cellular nuclei, thereby forming an immunohistological (IHC) sample comprising a plurality of nuclei. The method may further involve determining whether p53 staining in the IHC sample indicates abnormal p53 expression. For example, determining whether p53 staining in the IHC sample indicates abnormal p53 expression may include calculating the proportion of nuclei in the plurality of nuclei that has an intensity of p53 protein staining of at least 1 on a scale from 0 to 3.

In various embodiments, abnormal p53 expression was defined by both an upper and lower expression threshold as follows: 1) a proportion of nuclei having an intensity of 2-3+(e.g., 2+ or 3+) positivity of p53 protein staining above a threshold of at least 50% of cells, or 2) having no p53 protein staining at all (0+ staining in 100% of cells). The method may further comprise identifying the subject as at risk of developing dysplasia or esophageal cancer if p53 staining in the IHC sample indicates abnormal p53 expression.

In certain embodiments, the threshold is a proportion of nuclei having an intensity of 2+ or 3+ positivity of p53 protein staining above a threshold of 50% of cells (e.g., cell nuclei). In certain embodiments, the threshold is at a proportion of nuclei having an intensity of 2+ or 3+ positivity of p53 protein staining above a threshold of 50%, 51%, 52%, 53%, 54%, 55%, 56%, 57%, 58%, 59%, 60%, 65%, 70%, 75%, 80%, 85%, or 90% of cells (e.g., cell nuclei).

In some embodiments, the nuclei are in one or more (e.g., 1, 2, 3, 4, or 5) crypts. In various embodiments, the crypt or crypts are in a BE biopsy.

In certain embodiments, the proportion of nuclei with positive p53 staining may be rounded, e.g., to the nearest 1%, 5%, or 10%. In some implementations, the threshold yields a sensitivity of 96% and a specificity of 96% for differentiating non-dysplastic from high grade dysplastic samples, and is very strongly associated with the development of high grade dysplasia (P<0.0001).

In various embodiments, the p53 measured is or may be a mutated form (e.g., when a p53 protein is measured) or sequence (e.g., when a p53 mRNA or cDNA is measured) of p53. In some embodiments, the mutated form is a cancer-associated mutated form of p53. In certain embodiments, the p53 measured is or may be a wild-type form (e.g., when a p53 protein is measured) or sequence (e.g., when a p53 mRNA or cDNA is measured) of p53. In various embodiments, the p53 measured includes both a mutated form and a wild-type form (e.g., when a p53 protein is measured) or sequence (e.g., when a p53 mRNA or cDNA is measured) of p53.

Also provided is a method for identifying whether a subject who does not have dysplasia is at risk of developing dysplasia or esophageal cancer, comprising (a) providing a test sample from the subject; (b) assaying the level of p53 protein or p53-encoding mRNA in the test sample; and (c) identifying the subject as at risk of developing dysplasia or esophageal cancer if the level of p53 protein or p53-encoding mRNA in the test sample is elevated or reduced compared to a normal control.

In some embodiments, the subject is identified as at risk of developing dysplasia or esophageal cancer if the level of p53 protein or p53-encoding mRNA in the test sample is at least about 5%, 10%, 15%, 20%, 25%, 30%, 35%, 40%, 45%, 50%, 55%, 60%, 70%, 80%, 90%, 1-fold, 2-fold, 3-fold, 4-fold, 5-fold higher in the test sample compared to a normal control. In certain embodiments, the subject is identified as at risk of developing dysplasia or esophageal cancer if the level of p53 protein or p53-encoding mRNA in the test sample is reduced by at least about 5%, 10%, 15%, 20%, 25%, 30%, 35%, 40%, 45%, 50%, 55%, 60%, 70%, 80%, or 90% in the test sample compared to a normal control.

In various embodiments, the test sample comprises a biopsy such as a tissue biopsy, (e.g., an esophagus tissue biopsy). A non-limiting example of an esophagus tissue biopsy includes a BE biopsy. In some embodiments, the biopsy includes non-dysplastic tissue.

Techniques for assessing the level of p53 protein or mRNA include without limitation IHC, in situ hybridization (ISH), fluorescent in situ hybridization (FISH), various types of microarray (mRNA expression arrays, protein arrays, etc), various types of sequencing (Sanger, pyrosequencing, etc), comparative genomic hybridization (CGH), NextGen sequencing, Northern blot, Southern blot, immunoassay, reverse transcription polymerase chain reaction (RT-PCR), quantitative RT-PCR, and any other appropriate technique under development to assay the presence or quantity of a biological molecule of interest. Commonly used methods known in the art for the quantification of mRNA expression in a sample include northern blotting and in situ hybridization [Parker & Barnes (1999) Methods in Molecular Biology 106:247-283]; RNAse protection assays [Hod (1992) Biotechniques 13:852-854]; and reverse transcription polymerase chain reaction [Weis et al. (1992) Trends in Genetics 8:263-264]. Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). Any one or more of these methods can be used. In certain embodiments, more than one of any combination of these methods is concurrently or subsequent to each other. Non-limiting descriptions relating to the detection and quantitation of markers in samples, including p53, are provided in U.S. Pat. No. 9,389,234, issued Jul. 12, 2016 and U.S. Pat. No. 8,914,239, issued Dec. 16, 2014, the entire contents of each of which are incorporated herein by reference.

Embodiments of the present subject matter relate to prognostic and diagnostic tests for a subject who has BE. In some embodiments, the BE comprises (i) a circumferential extent of metaplasia that is less than about 0.25, 0.5, 0.75, 1, 1.25, 1.5, 2.0, 2.5, 3.0, 3.5, 4.0, 4.5, or 5 centimeters (cm); or at least about 0.25, 0.5, 0.75, 1, 1.25, 1.5, 2.0, 2.5, 3.0, 3.5, 4.0, 4.5, or 5 cm; and/or (ii) a maximum extend of metaplasia that is less than about 0.25, 0.5, 0.75, 1, 1.25, 1.5, 2.0, 2.5, 3.0, 3.5, 4.0, 4.5, or 5 cm or at least about 0.25, 0.5, 0.75, 1, 1.25, 1.5, 2.0, 2.5, 3.0, 3.5, 4.0, 4.5, or 5 cm. Alternatively, the subject does not have BE.

In certain embodiments, the subject is afflicted with gastroesophageal reflux disease. For example, the subject may have had gastroesophageal reflux disease for an extended period of time, such as at least about 1, 2, 3, 4, or 5 years. In embodiments, the subject suffers from heartburn, chronic cough, laryngitis, and/or nausea. In some embodiments, the subject has hiatal hernia, is at least 50 years of age, self-identifies as white or Caucasian, and/or is overweight.

Various embodiments of the present subject matter further include directing or advising the subject to obtain (i) additional screening or additional diagnostic testing for esophageal dysplasia or esophageal cancer; or (ii) treatment to reduce, delay, or prevent the onset or progression of dysplasia or esophageal cancer. In some embodiments, the subject is directed or advised to (i) eat less fatty food, chocolate, caffeine, spicy food, or peppermint; (ii) avoid alcohol, caffeinated beverages, or tobacco; or (iii) lose weight. In certain embodiments, the subject is administered a treatment such as (i) a proton pump inhibitor; (ii) an antacid; (iii) radiofrequency ablation (RFA); (iv) photodynamic therapy (PDT); (v) endoscopic spray cryotherapy; and/or (vi) endoscopic mucosal resection (EMR).

Aspects of the present subject matter relate to assessing a diagnostic risk or prognosis for dysplasia such as low-grade dysplasia or high-grade dysplasia.

In embodiments, the test sample comprises esophageal cells, and the level of p53 protein is the level of p53 protein in the nuclei of the esophageal cells. For example, the level of p53 protein in the nuclei of the esophageal cells may include (i) the proportion of nuclei having an amount of p53 protein that is 5%, 10%, 15%, 20%, 25%, 30%, 35%, 40%, 45%, 50%, 55%, 60%, 70%, 80%, 90%, 1-fold, 2-fold, 3-fold, 4-fold, 5-fold higher than the normal amount of p53 protein in an esophageal cell nucleus; or (ii) the proportion of nuclei having an amount of p53 protein that is least about 5%, 10%, 15%, 20%, 25%, 30%, 35%, 40%, 45%, 50%, 55%, 60%, 70%, 80%, or 90% lower than the normal amount of p53 protein in an esophageal cell nucleus.

Aspects of the present subject matter also provide a method for monitoring the development of dysplasia or esophageal cancer in a subject who has been diagnosed with BE but does not have dysplasia. In various embodiments, the method may involve periodically determining the level of p53 protein or p53-encoding mRNA in the subject, and identifying dysplasia or esophageal cancer as developing if the level of p53 increases or decreases over time. For example, determining the level of p53 protein or p53-encoding mRNA may comprise (a) providing a test sample from the subject; and (b) assaying the level of p53 protein or p53-encoding mRNA in the test sample. In embodiments, the level of p53 protein or p53-encoding mRNA is determined at least once every 1, 2, 3, 4, 6, 12, 18, or 24 months, or at least once every 3, 4, 5, 6, 7, 8, 9, or 10 years.

The present subject matter further includes a method for determining a prognosis for a subject who has been diagnosed with BE but does not have dysplasia. In embodiments, the method may include, e.g., (a) providing a test sample from the subject; (b) assaying the level of p53 protein or p53-encoding mRNA in the test sample; and (c) comparing the level of p53 protein or p53-encoding mRNA to a value in a database to identify the subject's risk of suffering from dysplasia or esophageal cancer. In a non-limiting example, the database may contain: (i) p53 protein or p53-encoding mRNA level values from subjects who have developed dysplasia or esophageal cancer; (ii) index values calculated based on p53 protein or p53-encoding mRNA levels in subjects who have developed dysplasia or esophageal cancer; (iii) p53 protein or p53-encoding mRNA level values from subjects who have developed dysplasia or esophageal cancer at various time points after the p53 protein or p53-encoding mRNA level values were provided from the subjects; (iv) index values calculated based on p53 protein or p53-encoding mRNA levels in subjects who have developed dysplasia or esophageal cancer at various time points after the p53 protein or p53-encoding mRNA level values were provided from the subjects; and/or (v) absolute or relative risk values calculated based on p53 protein or p53-encoding mRNA level values from subjects who have developed dysplasia or esophageal cancer. In some embodiments, the absolute or relative risk values comprise mean or median level values calculated using p53 protein or p53-encoding mRNA level values from subjects who have developed dysplasia or esophageal cancer.

In certain embodiments, assaying the level of p53 protein or p53-encoding mRNA comprises contacting p53 protein or p53-encoding mRNA in the test sample with a p53-specific binding agent. In embodiments, the binding agent may comprise an antibody or a fragment thereof. For example, the antibody may be an anti-p53 antibody. In some implementations, the p53-specific binding agent is attached to a solid support. In various embodiments, assaying comprises an enzyme-linked immunosorbent assay (ELISA), a radioimmunoassay, a fluoroimmunoassay, a Western blot, or immunohistochemistry (IHC).

In some embodiments, the binding agent comprises a primer, a pair of primers, or an oligonucleotide probe. For example, an assay may include a reverse transcriptase polymerase chain reaction (RT-PCR), quantitative PCT (qPCR), microarray analysis, or in situ hybridization.

In various embodiments, a primer or probe comprises DNA, RNA, or a hybrid thereof, or chemically modified analog or derivatives thereof. In certain embodiments, a primer or probe is single-stranded. However, they can also be double-stranded having two complementing strands which can be separated by denaturation. In some embodiments primers and probes have a length of from about 8 nucleotides to about 200 nucleotides, preferably from about 12 nucleotides to about 100 nucleotides, and more preferably about 18 to about 50 nucleotides. In certain embodiments, they can be labeled with detectable markers or modified using conventional manners for various molecular biological applications.

Aspects of the present subject matter further include a diagnostic system comprising, e.g., (a) an assortment, collection, or compilation of test results data scoring, representing, including, or corresponding to the level of p53 protein or p53-encoding mRNA in a plurality of test samples; (b) a means for computing an index value using the level, wherein the index value comprises a diagnostic, prognostic, or progression scores; and (c) a means for reporting the index value.

Also provided is a kit comprising a p53-specific binding agent for detecting the level of p53 protein or p53-encoding mRNA, and instructions for using the agent for determining whether a subject is at risk of developing dysplasia or esophageal cancer, for monitoring the progression from Barrett's esophagus (BE) to dysplasia or esophageal cancer, and/or for determining the prognosis of the subject.

All references to “dysplasia” herein refer to esophageal dysplasia.

Included herein are methods for treating a subject who has been identified as at risk of developing dysplasia or esophageal cancer. In various embodiments, radiofrequency ablation (such as balloon-based radiofrequency ablation) is administered to a subject with BE who is identified as at risk of developing dysplasia. In some embodiments, a proton pump inhibitor or a nonsteroidal anti-inflammatory drug (NSAID) such as aspirin is administered to a subject. In certain embodiments, the subject receives laser treatment, surgery, endoscopic mucosal resection, or Nissen fundoplication.

The methods described herein may also include measuring or computing a level of p53 protein or p53-encoding mRNA with a binding agent. Exemplary examples of a binding agent comprise an antibody (or fragment thereof), a detectable protein (or fragment thereof), or any combination thereof. The antibody may be labeled with a detectable moiety, e.g., a fluorescent compound [e.g., Alexa 350, Alexa 430, aminomethylcoumarin (AMCA), BODIPY 630/650, BODIPY 650/665, boron-dipyrromethene (BODIPY)-FL, BODIPY-R6G, BODIPY-TMR, BODIPY-TRX, Cascade Blue, Cy3, Cy5, 6-Carboxyfluorescein (6-FAM), Fluorescein Isothiocyanate, hexachloro-fluorescein (HEX), 6-carboxy-4′,5′-dichloro-2′,7′-dimethoxyfluorescein (6-JOE), Oregon Green 488, Oregon Green 500, Oregon Green 514, Pacific Blue, REG, Rhodamine Green, Rhodamine Red, Renographin, ROX, TAMRA (e.g., 5-carboxytetramethylrhodamine), TET, Tetramethylrhodamine, and/or Texas Red] or a radioactive agent (e.g., astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium⁹⁹ⁿ¹ and/or yttrium⁹⁰). When the fluorescently labeled antibody is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, p-phthaldehyde and fluorescamine. The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA). The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt, and oxalate ester.

In various embodiments, specific binding agent has greater than 10-fold, preferably greater than 100-fold, and most preferably, greater than 1000-fold affinity for the target molecule compared to another molecule. The term “specific” is used to indicate that other biomolecules present in the sample do not significantly bind to the binding agent that is specific for the target molecule. Preferably, the level of binding to a biomolecule other than the target molecule results in a binding affinity which is at most only 10% or less, only 5% or less, only 2% or less, only 1% or less, or less than 1% of the affinity to the target molecule. A preferred specific binding agent will fulfill both the above minimum criteria for affinity as well as for specificity. For example, an antibody may have a binding affinity in the low micromolar (10⁻⁶) or nanomolar (10⁻⁹) range, with high affinity antibodies in the low nanomolar (10⁻⁹) or picomolar (10⁻¹²) range for its specific target ligand.

Various implementations of the present subject matter include a composition comprising a binding agent, wherein the binding agent is attached to a solid support, (e.g., a strip, a polymer, a plate such as a multiwell plate, a nanoparticle, or a microparticle). Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble. The support material may have virtually any possible structural configuration so long as the test molecule is capable of binding to the binding agent (e.g., an antibody). Thus, the support configuration may be spherical, as in a bead, or cylindrical, as in the inside surface of a test tube, or the external surface of a rod. Alternatively, the surface may be flat such as a sheet, plate, or test strip. Exemplary supports include polystyrene beads. Those skilled in the art will know many other suitable carriers for binding antibody or antigen, or will be able to ascertain the same by use of routine experimentation.

In embodiments, the solid support comprises a polymer, to which an agent is chemically bound, immobilized, dispersed, or associated. A polymer support may be a network of polymers, and may be prepared in bead form (e.g., by suspension polymerization). The location of active sites introduced into a polymer support depends on the type of polymer support. For example, in a swollen-gel-bead polymer support the active sites are distributed uniformly throughout the beads, whereas in a macroporous-bead polymer support they are predominantly on the internal surfaces of the macropores. The solid support, e.g., a device, may contain a p53-specific binding agent.

The term “sample” as used herein refers to a biological sample obtained for the purpose of evaluation in vitro. With regard to the methods provided herein, the sample or patient sample may comprise, e.g., a biopsy of esophagus tissue, such as BE tissue.

Optionally, the method further comprises repeating the providing, contacting, detecting, and computing steps over time. A progressive change (e.g., decrease or increase) over time in the level of p53 protein and/or p53-encoding mRNA indicates a progression and/or development of dysplasia or esophageal cancer. Optionally, the method may also include the step of risk stratification and/or treatment following a diagnostic method as described above. For example, the method further comprises identifying a subject with a high risk of dysplasia or esophageal cancer and administering to that subject a treatment to inhibit, prevent, or treat dysplasia or esophageal cancer.

Also provided is a kit comprising a p53 protein and/or p53-encoding mRNA binding agent and instructions for using the agent for evaluating the risk of dysplasia or esophageal cancer. In some embodiments, the agent is attached to a solid support. The kit optionally contains buffers, enzymes, salts, stabilizing agents, preservatives, and/or a container for receiving a patient test sample of bodily fluid or cell. In some cases such a container contains an anti-coagulant, cell separation agent, and/or a cell lysis reagent, e.g., to liberate a p53 protein and/or p53-encoding mRNA from cells to permit measurement of the protein or mRNA. In various embodiments, a kit comprises agents for measuring a plurality of markers, where the plurality of markers includes p53. In some variations, such agents are packaged together. In some variations, the kit further includes an analysis tool for evaluating risk of an individual developing dysplasia or esophageal cancer from measurements of p53 protein and/or p53-encoding mRNA from at least one biological sample from the subject.

The diagnostic or prognostic assay is optionally formulated in a two-antibody binding format in which one p53 protein-specific antibody captures a p53 protein derived from (e.g., isolated from) a patient sample and another antibody (e.g., an anti-IgG antibody or a second anti-p53 antibody) is used to detect captured protein. For example, the capture antibody is immobilized on a solid phase, e.g., an assay plate, an assay well, a nitrocellulose membrane, a bead, a dipstick, or a component of an elution column. The second antibody, i.e., the detection antibody, is typically tagged with a detectable label such as a colorimetric agent or radioisotope.

Also provided is a diagnostic test system that obtains test results data representing levels of p53 protein and/or p53-encoding mRNA in at least one biological sample. The results are collected and tracked and an index value is calculated from said marker, wherein the index value comprises a dysplasia risk score or an esophageal cancer risk score. The test system may further comprise a means of reporting the index value. An exemplary diagnostic test system is a system for obtaining test results data representing levels of one or more markers (e.g., p53) in at least one biological sample comprising (i) a means for collecting and tracking test results data for one or more individual biological samples; (ii) a means for computing an index value from marker measurement data, wherein said biomarker measurement data is representative of measured levels of a marker; and (iii) a means for reporting said index value. In some variations of the diagnostic test system, the index value indicates a dysplasia or esophageal cancer risk score. In some variations, the dysplasia or esophageal cancer risk score is computed according to a method described herein for computing such scores. In some embodiments, the means for collecting and tracking test results data representing for one or more individuals comprises a data structure or database. In some variations, the means for computing a dysplasia or esophageal cancer risk score comprises a computer or microprocessor, comprising a visible display, an audio output, a link to a data structure or database, and/or a printer.

In various embodiments, the practice of the present subject matter may employ conventional biology methods, software and systems. Computer software products provided herein typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present subject matter may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention relates to embodiments that include methods for providing genetic information over networks such as the Internet as shown in U.S. Ser. Nos. 10/197,621, 10/063,559 (U.S. Publication Number 20020183936), Ser. Nos. 10/065,856, 10/065,868, 10/328,818, 10/328,872, 10/423,403, and 60/482,389. For example, one or more molecular profiling techniques can be performed in one location, e.g., a city, state, country or continent, and the results can be transmitted to a different city, state, country or continent. Treatment selection can then be made in whole or in part in the second location. The methods of the invention comprise transmittal of information between different locations.

Each embodiment disclosed herein is contemplated as being applicable to each of the other disclosed embodiments. Thus, all combinations of the various elements described herein are within the scope of the invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a bar graph showing 3+ p53 expression in Barrett's biopsies. Bars on the left at each position on the X axis are for non-progressors. Bars on the right at each position on the X axis correspond to simultaneous with progression.

FIG. 2 is a bar graph showing 2-3+ p53 expression in Barrett's biopsies. Bars on the left at each position on the X axis are for non-progressors. Bars on the right at each position on the X axis correspond to simultaneous with progression.

FIG. 3 is a flow chart showing a non-limiting example of a process for detecting abnormal p53 expression.

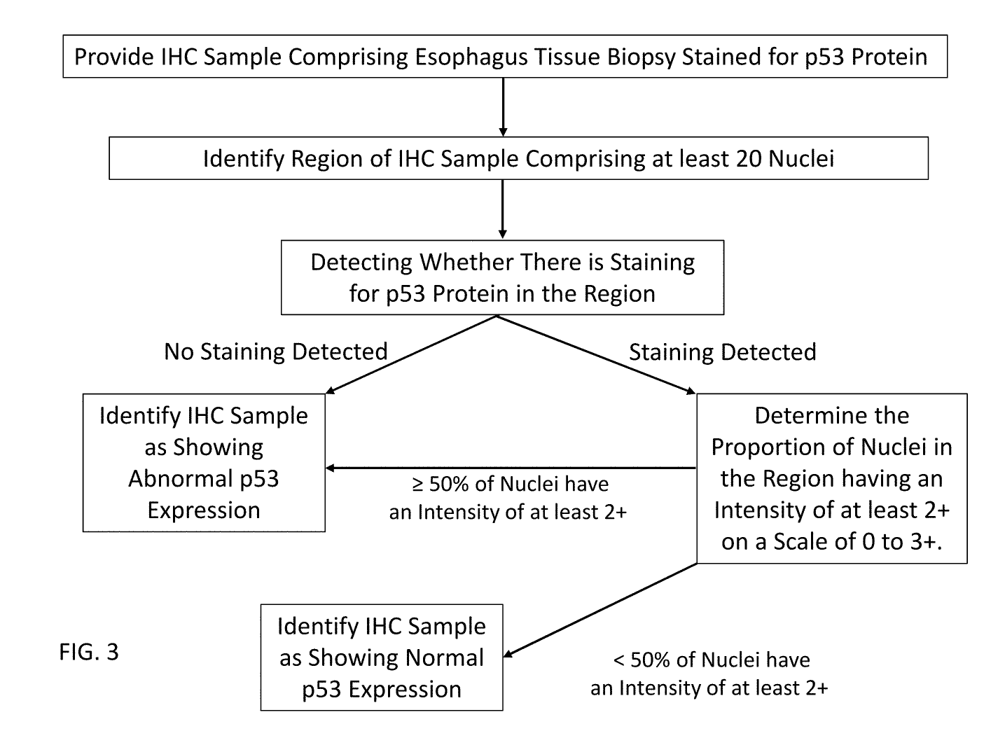

FIG. 4 is an image showing an illustrative example of immunohistochemical scoring of p53 expression in individual Barrett's epithelium cells. Expression of p53 was scored in individual Barrett's epithelium cell nuclei as follows: 0+ (blue arrows)=no staining in nuclei, which appear light blue from the counter stain; 1+ (light brown arrows)=faint brown staining, only apparent using 200× magnification or higher; 2+ (medium brown arrows)=medium brown staining, readily apparent at 100× magnification; 3+ (dark brown arrows)=dark brown staining, readily apparent at 20× magnification. Original magnification 200×.

FIG. 5 is an image and a table showing an illustrative example of scoring of p53 expression in Barrett's biopsies. The entire biopsy is evaluated, and expression is scored in regions with the highest overall expression. In this example, three separate crypts are highlighted (A, B and C), and the number of cells with nuclear expression levels of 0+ to 3+ are counted (Table). The overall score is calculated as the percent of cells in a crypt (or contiguous focus of at least 20 cells) with 2-3+ nuclear positivity. In the three highlighted crypts, the percentage of cells with 2-3+ positivity is 7%, 5% and 9%, well below the established threshold for an abnormal result of >50% and consistent with non-mutated (wild-type) p53. Original magnification 200×.

FIG. 6 is an image and a table showing an illustrative example of abnormal high level of p53 expression in a patient that subsequently progressed to adenocarcinoma. Most of the crypts to the left of the field have relatively low p53 nuclear expression. One representative crypt, A, has a score of 9%, consistent with wild-type (non-mutated). A single crypt to the left of the field, B, has somewhat higher p53 expression, but a score of 43% is still below the abnormal threshold (>50%). In contrast, the crypts to the right of the field appear to have very high p53 nuclear expression, and the score of 81% in a single representative crypt, C, is well above the threshold for abnormal (mutated) p53. Original magnification 200×.

FIG. 7 is an image and a table showing an illustrative example of abnormal (absent) p53 expression in a patient that subsequently progressed to adenocarcinoma. A few crypts have non-mutated (wild-type) p53 expression, for example, the p53 score in representative crypt A is 7%, well below the threshold of >50%. Importantly, most cells have some p53 expression (expected), and only 9% of cell nuclei have 0+ staining. In contrast, there are several other crypts with no p53 expression. The p53 score in representative crypt B reveals 0+ staining in 100% of cells. This absence of p53 expression is the second abnormal (null mutation) pattern identified by our methods. Original magnification 200×.

FIG. 8 is an image showing an illustrative example in which 2-3+ nuclear staining is present in 7% of crypt epithelial nuclei.

FIG. 9 is an image showing an illustrative example in which 2-3+ nuclear staining is present in 100% of crypt base epithelial nuclei.

### DETAILED DESCRIPTION OF THE DISCLOSURE

The present subject matter provides improvements over existing methodologies for evaluating the risk of developing dysplasia and/or esophageal cancer in a subject. Aspects of the present disclosure relate to the surprising discovery that p53 mutations are vital to neoplastic progression of most BE associated carcinomas, and that these mutations often occur prior to the development of dysplasia. The potential use of p53 IHC has not been properly assessed during the early stages of progression from BE to cancer. For example, the threshold for an abnormal p53 IHC stain has not been systematically studied in BE biopsies, and the frequency of abnormal p53 IHC in non-dysplastic BE has not been determined.

Aspects of the present subject matter provide a p53 IHC test, to be performed on BE biopsies without dysplasia, that is highly predictive of the subsequent development of high grade dysplasia or cancer. In certain embodiments, a positive test is associated with an approximate 20-fold increased risk of progression to high grade dysplasia or cancer. Importantly, in various implementations, the p53 IHC tests are positive more than five years prior to the development of high grade dysplasia or cancer. The p53 IHC test provided herein is extremely valuable for identifying patients at high risk for progression to high grade dysplasia or cancer, and will have a major impact on BE screening.

Mutations in p53 result in alterations in the quantity of p53 protein expressed in human cells. Therefore, p53 IHC can be utilized as a surrogate marker of underlying p53 mutation, allowing for a potentially inexpensive assay that can be readily applied to small biopsy samples. Unfortunately, previous thresholds for determining normal versus abnormal p53 IHC expression have been based primarily on empiric cut-offs. A sampling of non-dysplastic and high grade dysplastic BE biopsies can be used to determine an optimal threshold for abnormal p53 staining.

The present disclosure includes two unique aspects: First, a p53 IHC test has been developed that includes a threshold for p53 that is predictive and specific for increased risk of dysplasia and esophageal cancer. The p53 IHC test has very high sensitivity and specificity for identifying p53 abnormalities. Second, unique tissue resources were used to assemble the largest case-control study ever undertaken in BE. Surprisingly, p53 IHC testing in patients with non-dysplastic BE can identify a high risk subgroup of patients who will progress to high grade dysplasia or carcinoma. The methods, compositions, and devices provided herein have useful advantages for the screening and management of patients with BE.

The normal esophagus does not have any intestinal epithelium (crypts). In BE, the normal esophageal squamous epithelium is replaced by epithelium that appears morphologically similar to intestinal epithelium, which has crypts (epithelial invaginations beneath the surface) as well as surface epithelium.

In various embodiments, methods provided herein score only the intestinal epithelium, which includes the crypts and surface epithelium. Expression of p53 is confined to the nuclei, and only scoring nuclear staining is scored.

The 0-3+ scoring system is integer only. The “+” sign is commonly used in pathology literature to describe positive staining. However, as used herein, the “+” sign may optionally be omitted. Therefore, as used herein with respect to a staining value, use 0, 1, 2, and 3 are interchangeable with 0+, 1+, 2+, and 3+, respectively.

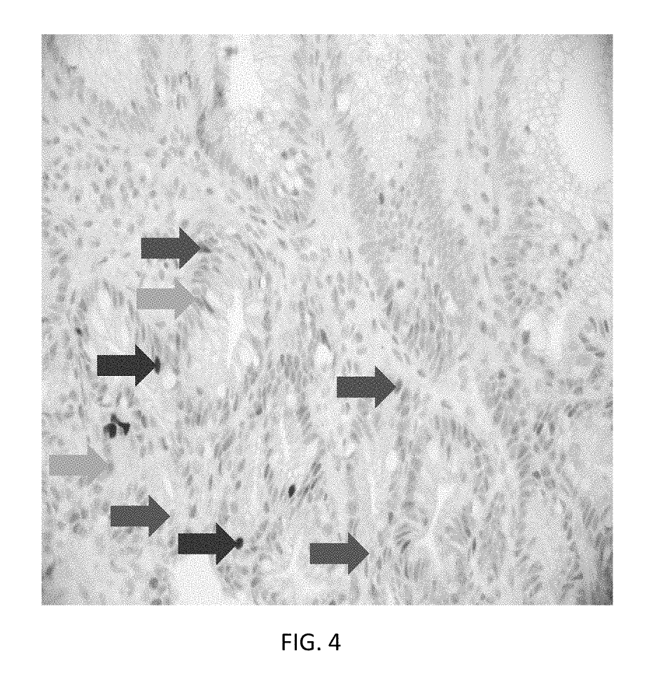

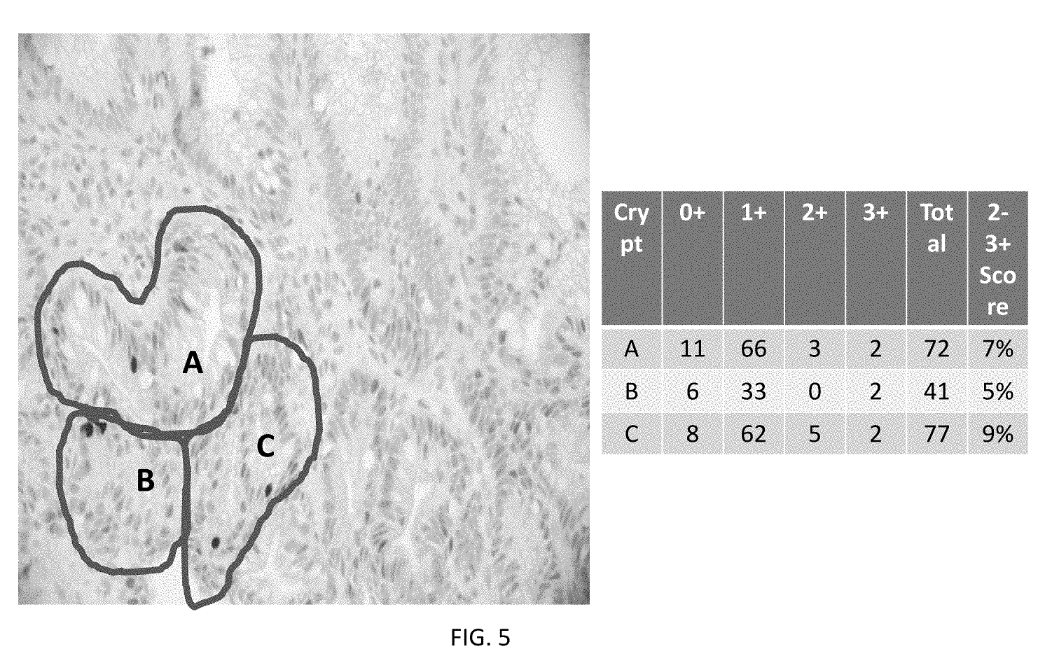

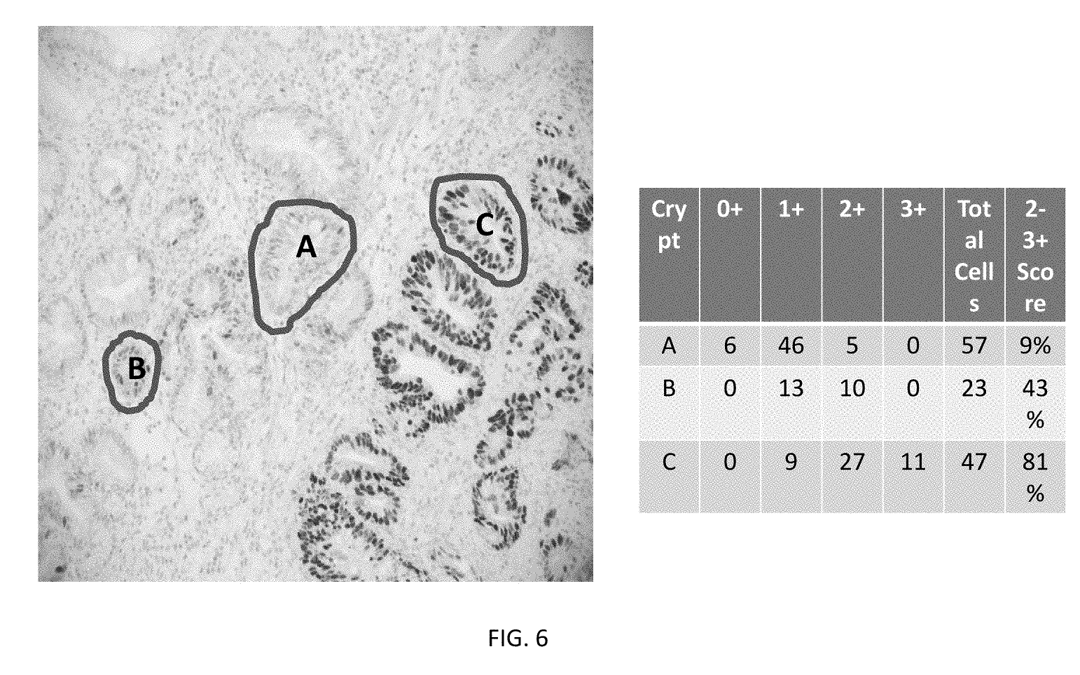

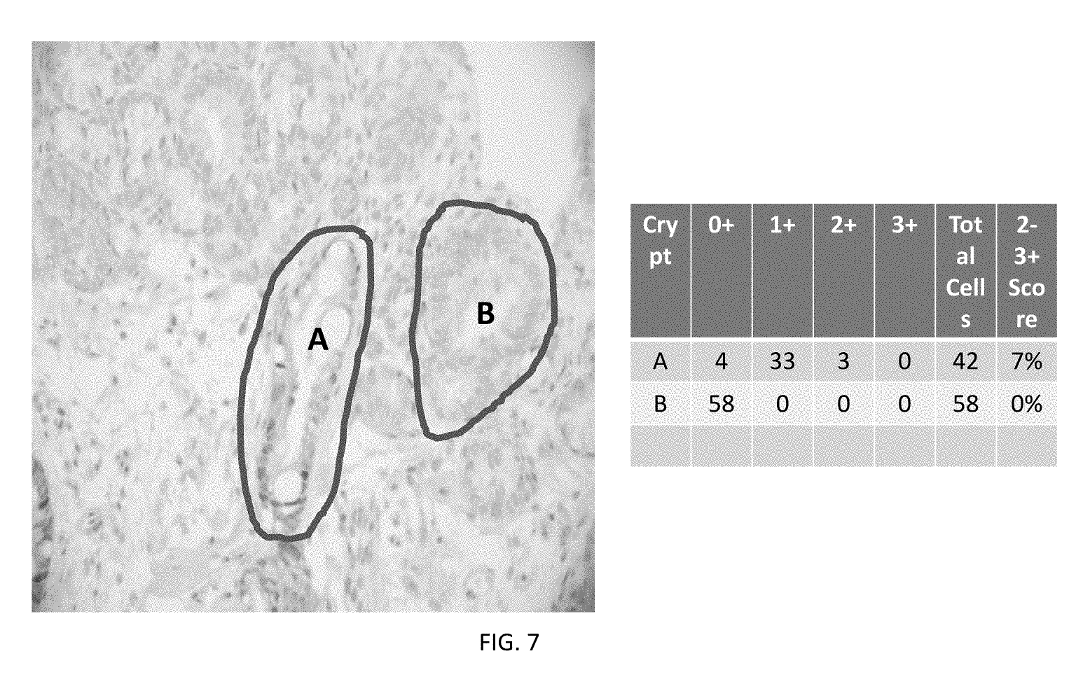

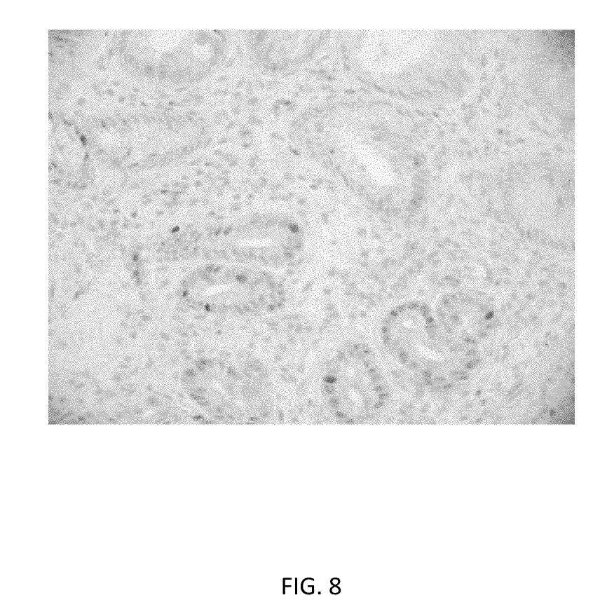

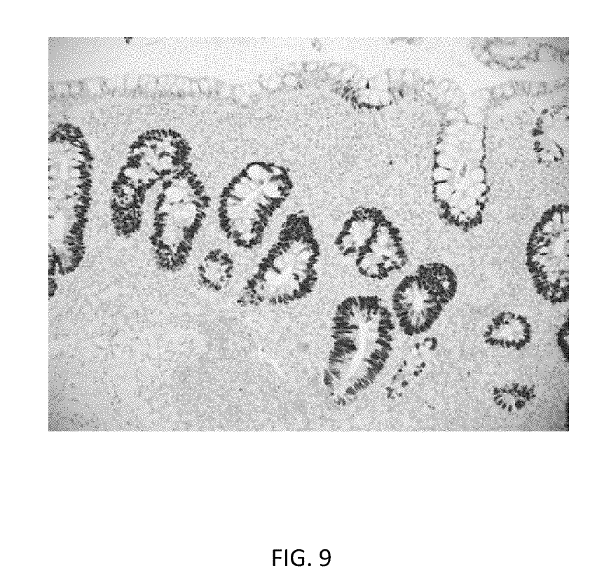

The 0-3+ system is commonly used in pathology literature and is based on a quantitation defined as follows: 0=no staining, 1+=weak signal intensity, 2+=medium signal intensity, and 3+=strong signal intensity. In some embodiments, the quantitation is defined as follows: 0=no staining, 1+=light brown, 2+=medium brown, and 3+=dark brown (depending on, e.g., the color of the stain used). The intensity of other signals (such as other stain colors) may similarly be quantified into 0+, 1+, 2+, or 3+ values. The quantitation of a signal (such as from p53 staining) into 0+, 1+, 2+, and 3+ values is commonly used and well within the skill of a pathologist. Additionally, the scoring methods and thresholds provided herein take into account and reduce or eliminate the effects of subjective variability. In certain embodiments: (i) a value of 3+ is the strongest signal in a sample (e.g., an image of a sample) or a signal having at least 80% (e.g., at least 80%, 85%, 90%, or 95%) of the signal strength or intensity of the strongest signal in the sample; (ii) a value of 0+ is no signal; (iii) a value of 1+ is a weak signal that is up to 30% (e.g., more than 0% or no signal but less than 30%, 25%, or 20%) the strength or intensity of 3+; and (iii) a value of 2+ is a signal that is stronger than 1+ but weaker than 3+. All biopsies from BE subjects have admixed normal cells (stromal cells, lymphocytes and often squamous epithelium, all unrelated to the BE) that serve as an internal control to gauge that the stain is working correctly. In various embodiments, the p53 score is determined by identifying the region of the biopsy with strongest staining, and evaluating at least 20 contiguous crypt epithelial cells, and determining the percentage of cells that have 2-3+ positivity. The highest score is generally found in the crypt base; however, if staining is more intense at the surface, any 20 contiguous cells can be used to obtain a score. Illustrative examples of 0+, 1+, 2+, and 3+ staining are provided in, e.g., FIGS. 4-10.

In some embodiments, the scoring of immunohistochemistry images is automated. Non-limiting descriptions relating to the automated analysis of images are provided in, e.g., Theodosiou et al. (2007) Cytometry Part A 71; 7:439-50; and Rizzardi et al. (2012) Diagnostic Pathology 7:42, the entire contents of each of which are incorporated herein by reference. In certain embodiments, the scoring is performed manually by as pathologist.

### Barrett's Esophagus

BE is also known as Barrett syndrome, Barrett esophagus, and columnar epithelium lined lower oesophagus (CELLO). BE is an abnormal change (metaplasia) in the cells of the lower portion of the esophagus. It is characterized by the replacement of the normal stratified squamous epithelium lining of the esophagus by simple columnar epithelium with goblet cells (similar to those found lower in the gastrointestinal tract). BE is strongly associated (about 0.5% per patient-year) with esophageal adenocarcinoma, a deadly cancer, and is considered to be a premalignant condition.

Without wishing to be bound by any scientific theory, the main cause of BE is thought to be an adaptation to chronic acid exposure from reflux esophagitis. The condition is found in 5-15% of patients who seek medical care for heartburn (gastroesophageal reflux disease), although a large subgroup of patients with BE do not have symptoms. The cells of BE, after biopsy, are classified into four general categories: nondysplastic, low-grade dysplasia, high-grade dysplasia, and frank carcinoma. High-grade dysplasia and early stages of adenocarcinoma can be treated by endoscopic resection and endoscopic therapies such as radiofrequency ablation, whereas advanced stages of adenocarcinoma (submucosal) are generally advised to undergo surgical treatment. Nondysplastic and low-grade patients are generally advised to undergo annual observation with endoscopy, with radiofrequency ablation as a therapeutic option. In high-grade dysplasia, the risk of developing cancer might be 10% per patient-year or greater.

The change from normal to premalignant cells that indicate BE does not cause any particular symptoms. However, BE is associated with frequent and longstanding heartburn, trouble swallowing (dysphagia), vomiting blood (hematemesis), pain under the sternum where the esophagus meets the stomach, and unintentional weight loss due to pain felt while eating (odynophagia). The risk of developing BE is increased by central obesity (vs. peripheral obesity). The exact mechanism is unclear. The difference in distribution of fat among men (more central) and women (more peripheral) may explain an increased risk in males.

Without wishing to be bound by any scientific theory, BE occurs due to chronic inflammation. The principal cause of the chronic inflammation is gastroesophageal reflux disease (GERD). In this disease, acidic stomach, bile, and/or small intestine and pancreatic contents cause damage to the cells of the lower esophagus. Researchers have been unable to predict which heartburn sufferers will develop BE. While no relationship exists between the severity of heartburn and the development of BE, a relationship does exist between chronic heartburn and the development of BE. Sometimes, people with BE have no heartburn symptoms at all. In rare cases, damage to the esophagus may be caused by swallowing a corrosive substance such as lye.

Many people with BE do not have dysplasia. Endoscopic surveillance of people with BE is often recommended, although little direct evidence supports this practice. Treatment options for high-grade dysplasia include surgical removal of the esophagus (esophagectomy) or endoscopic treatments such as endoscopic mucosal resection or ablation (destruction).

The risk of malignancy is particularly high in Caucasian men more than 50 years of age with more than five years of symptoms. Current recommendations include routine endoscopy and biopsy (looking for dysplastic changes).

### Immunohistochemistry

IHC is a process of localizing antigens (e.g., proteins) in cells of a tissue comprising binding antibodies specifically to antigens in the tissues. In various embodiments, the antigen-binding antibody can be conjugated or fused to a tag that allows its detection, e.g., via visualization. In some embodiments, the tag is an enzyme that can catalyze a color-producing reaction, such as alkaline phosphatase or horseradish peroxidase. In certain embodiments, the enzyme can be fused to the antibody or non-covalently bound, e.g., using a biotin-avadin system. Alternatively, the antibody can be tagged with a fluorophore, such as fluorescein, rhodamine, DyLight Fluor or Alexa Fluor. In various embodiments, the antigen-binding antibody can be directly tagged or it can itself be recognized by a detection antibody that carries the tag. Using IHC, proteins such as p53 may be detected. The expression of a gene product can be related to its staining intensity compared to control levels. In some embodiments, the gene product is considered differentially (e.g., abnormally) expressed if its staining varies at least 1.2, 1.3, 1.4, 1.5, 1.6, 1.7, 1.8, 1.9, 2.0, 2.2, 2.5, 2.7, 3.0, 4, 5, 6, 7, 8, 9 or 10-fold in the sample versus the control.

IHC combines anatomical, immunological and biochemical techniques to identify discrete tissue components by the interaction of target antigens with specific antibodies tagged with a detectable label. IHC makes it possible to visualize the distribution and localization of specific cellular components within cells and in the proper tissue context. There are multiple approaches and permutations of IHC methodology. However, IHC procedures can typically be placed into one of two groups: sample preparation and labeling. Steps relating to sample preparation generally include tissue collection (e.g., biopsy), tissue fixation, tissue embedding, sectioning and mounting, epitope recovery, quenching/blocking endogenous target activity, and blocking nonspecific sites. Steps relating to labeling typically include immunodetection, counterstaining, and sealing the stained sample. Non-limiting aspects of these steps are discussed below.

IHC is used for, e.g., disease diagnosis, drug development and biological research. For example, using specific tumor markers, physicians have used IHC to diagnose a cancer as benign or malignant, determine the stage and grade of a tumor, and identify the cell type and origin of a metastasis to find the site of the primary tumor. IHC is also used in drug development to test drug efficacy by detecting either the activity or the up-regulation or down-regulation of disease targets.

In various implementations, samples are prepared on individual slides, or multiple samples can be arranged on a single slide for comparative analysis, such as with tissue microarrays. IHC slides can be processed and stained manually, however technological advances provide automation for high-throughput sample preparation and staining. Samples can be viewed by either light or fluorescence microscopy. Many devices and methods are available for capturing images, quantitating multiparametric IHC data, and increasing the collection of that data through high content screening.

Tissue Collection and Perfusion

Patient or animal biopsies, or whole animal organs, are collected for preservation and IHC analysis, depending on the requirements of the assay. Tissue should be rapidly preserved to prevent the breakdown of cellular protein and tissue architecture. In some embodiments, the tissue is perfused, or rinsed of blood, prior to preservation to prevent the detection of hematologic antigens that may interfere with the detection of target antigens.

Tissue Fixation

Fixation chemically crosslinks proteins or reduces protein solubility, which can mask target antigens during prolonged or improper fixation. Therefore, the right fixation method should be optimized based on the application and the target antigen to be stained.

A common fixative is formaldehyde, a semi-reversible, covalent crosslinking reagent that can be used for perfusion or immersion fixation for any length of time, depending on the level of fixation required. Other fixatives are available, and their use depends on the antigens that are being sought.

Tissue Embedding

In various embodiments, fixed tissue samples are embedded in paraffin to maintain the natural shape and architecture of the sample during long-term storage and sectioning for IHC. Samples too sensitive for either chemical fixation or the solvents used to remove the paraffin may be encased in cryogenic embedding medium and then snap-frozen in liquid nitrogen.

Sectioning and Mounting

The decision to section tissue is dependent upon the application used; for example, whole mount IHC, with sample thickness up to 5 mm, does not require sectioning, while small samples for which multiple staining procedures are needed may require sectioning.

In certain embodiments, formalin-fixed, paraffin-embedded tissues are sectioned into thin slices (e.g., about 4, 5, 6, 7, 8, 9, 10, 25, 50, 100, or 1-100 μm) with, e.g., a microtome. In some embodiments, these sections are then mounted onto slides that are coated with an adhesive. This adhesive is commonly added by surface-treating glass slides with 3-aminopropyltriethoxysilane (APTS) or poly-L-lysine, which both leave amino groups on the surface of the glass to which the tissue directly couples. In some embodiments, slides may be coated with physical adhesives, including gelatin, egg albumin, or Elmer's glue. In certain embodiments, after mounting, the sections are dried in an oven or microwave in preparation for deparaffinization.

In embodiments, frozen sections are cut using a pre-cooled cryostat and mounted to adhesive slides, such as glass slides. In some embodiments, these sections are dried overnight at room temperature and fixed by immersion in pre-cooled (e.g., −20° C.) acetone, although the drying step may be skipped.

Epitope (Antigen) Recovery

In some embodiments, the paraffin from formalin-fixed, paraffin-embedded sections is completely removed so that the antibodies can reach target antigens. Xylene, a flammable, toxic and volatile organic solvent is commonly used to remove the paraffin from IHC slides, although commercial alternatives are available.

Formaldehyde fixation generates methylene bridges that crosslink proteins in tissue samples; these bridges can mask antigen presentation and prevent antibody binding. Formalin-fixed, paraffin-embedded sections commonly require a treatment to unmask the antibody epitopes, either by heat (heat-induced epitope retrieval; HIER) or enzymatic degradation (proteolytic-induced epitope retrieval; PIER).

Quenching/Blocking Endogenous Target Activity

For staining approaches that depend on biotin, peroxidases or phosphatases for the amplification or enzymatic detection of target antigens, quenching or masking endogenous forms of these proteins prevents false positive and high background detection. The general strategies include physically blocking or chemically inhibiting all endogenous biotin or enzyme activity, respectively.

Blocking Nonspecific Sites

Although antibodies show preferential avidity for specific epitopes, antibodies may partially or weakly bind to sites on nonspecific proteins (also called reactive sites) that are similar to the cognate binding sites on the target antigen. In the context of antibody-mediated antigen detection, nonspecific binding causes high background staining that can mask the detection of the target antigen. To reduce background staining in IHC or any other immunostaining application, the samples are incubated with a buffer that blocks the reactive sites to which the primary or secondary antibodies may otherwise bind. Common blocking buffers include normal serum, non-fat dry milk, bovine serum albumin (BSA), or gelatin, and commercial blocking buffers with proprietary formulations are available for greater efficiency.

Immunodetection

Detecting the target antigen with antibodies is typically a multi-step process that may require optimization to maximize the signal detection. Both primary and secondary antibodies may be diluted into a buffer to help stabilize the antibody, promote the uniform dissemination throughout the sample and discourage nonspecific binding. While one diluent may work with one antibody, the same diluent may not work with another antibody, demonstrating the need for optimization for each antibody.

In some embodiments, the sample is rinsed between antibody applications to remove unbound antibodies and also to remove antibodies that are weakly bound to nonspecific sites. Rinse buffers are usually simple solutions of only a few components, but the right components should be considered to maximize sample washing and minimize interference with the signal detection.

In various aspects, antibody-mediated antigen detection approaches are separated into direct and indirect methods. These methods both use antibodies to detect the target antigen, but the selection of the best method to use depends on the level of target antigen expression and availability and also the readout desired. Some embodiments involving indirect methods employ the inherent binding affinity of avidin to biotin to localize a reporter to the target antigen and amplify the signal that is detected.

In certain embodiments, IHC target antigens are detected through either chromogenic or fluorescent means. In some embodiments relating to fluorescent detection, the reporter that the primary or secondary antibody is conjugated to is a fluorophore that is detected by fluorescent microscopy. In some embodiments relating to chromogenic detection, detection is based on the activates of an enzyme such as horseradish peroxidase (HRP) or alkaline phosphatase (AP), which form colored, insoluble precipitates upon the addition of substrate, such as 3,3′-diaminobenzidine (DAB) and nitro-blue tetrazolium chloride (NBT)/5-bromo-4-chloro-3-indolyl-phosphate (BCIP), respectively.

Counterstaining

Counterstains give contrast to the primary stain and can be cell structure-specific. In various embodiments, single-step stains are added after antibody staining Non-limiting examples of chromogenic counterstains include hematoxylin, nuclear fast red, and methyl green. Non-limiting examples of fluorescent counterstains include DAPI (4′, 6-diamidino-2-phenylindole), propidium iodide, and phalloidin.

Sealing the Stained Sample

After all staining is completed, the sample may be preserved for long-term usage and storage and to prevent enzymatic product solubilization or fluorophore photobleaching. Sealing the sample by mounting a coverslip with an appropriate mountant stabilizes the tissue sample and stain. An antifade reagent may also be included if fluorescent detection will be performed to prolong fluorescence excitation. A coverslip can then be sealed with clear nail polish or a commercial sealant after the mountant has cured to prevent sample damage.

### p53 as a Biomarker of BE Progression

Several studies have attempted to determine whether p53 alterations could be used to predict progression risk in BE patients with dysplasia. While these studies have revealed some potential for p53 as a biomarker, clinical application has been limited by the small size of most studies, and by a number of technical challenges, including the difficulty and expense of sequencing p53 in small biopsy samples, the low sensitivity of assays of genomic deletion of p53, and the interpretative variability and lack of established thresholds for evaluating p53 IHC in BE. There are no large studies examining the role of p53 as a biomarker in BE patients with no dysplasia.

### p53 in Human Cancer

Most human cancers, including esophageal cancer, are characterized by an accumulation of genomic alterations, as well as mutations in oncogenes and tumor suppressor genes. Amongst the hundreds of genes that are mutated in human cancers, alterations in p53, a gene involved in DNA damage response and cell cycle regulation, are the most common.

### p53 in Barrett's Esophagus

Inactivating p53 mutations and genomic deletions of p53 are the most common genetic alterations in esophageal adenocarcinomas, and are present in approximately 50-70% of these cancers. Alterations in p53 have also been documented in 40-60% of high grade dysplasia and 30-50% of low grade dysplasia. Alterations in p53 have not been fully characterized in non-dysplastic BE.

### Tumor Protein p53

The gene for p53 is also known as Tumor protein p53 (TRP53). A nucleotide sequence that encodes human p53 is publically available in the GenBank database under accession number AB082923 (SEQ ID NO: 1) and is provided below. The start and stop codons of the coding sequence are bolded and underlined.

CGTGCTTTCC ACGACGGTGA CACGCTTCCC TGGATTGGCC
AGACTGCCTT CCGGGTCACT GCCATGGAGG AGCCGCAGTC
AGATCCTAGC GTCGAGCCCC CTCTGAGTCA GGAAACATTT
TCAGACCTAT GGAAACTACT TCCTGAAAAC AACGTTCTGT
CCCCCTTGCC GTCCCAAGCA ATGGATGATT TGATGCTGTC
CCCGGACGAT ATTGAACAAT GGTTCACTGA AGACCCAGGT
CCAGATGAAG CTCCCAGAAT GCCAGAGGCT GCTCCCCGCG
TGGCCCCTGC ACCAGCAGCT CCTACACCGG CGGCCCCTGC
ACCAGCCCCC TCCTGGCCCC TGTCATCTTC TGTCCCTTCC
CAGAAAACCT ACCAGGGCAG CTACGGTTTC CGTCTGGGCT
TCTTGCATTC TGGGACAGCC AAGTCTGTGA CTTGCACGTA
CTCCCCTGCC CTCAACAAGA TGTTTTGCCA ACTGGCCAAG
ACCTGCCCTG TGCAGCTGTG GGTTGATTCC ACACCCCCGC
CCGGCACCCG CGTCCGCGCC ATGGCCATCT ACAAGCAGTC
ACAGCACATG ACGGAGGTTG TGAGGCGCTG CCCCCACCAT
GAGCGCTGCT CAGATAGCGA TGGTCTGGCC CCTCCTCAGC
ATCTTATCCG AGTGGAAGGA AATTTGCGTG TGGAGTATTT
GGATGACAGA AACACTTTTC GACATAGTGT GGTGGTGCCC
TATGAGCCGC CTGAGGTTGG CTCTGACTGT ACCACCATCC
ACTACAACTA CATGTGTAAC AGTTCCTGCA TGGGCGGCAT
GAACCGGAGG CCCATCCTCA CCATCATCAC ACTGGAAGAC
TCCAGTGGTA ATCTACTGGG ACGGAACAGC TTTGAGGTGC
ATGTTTGTGC CTGTCCTGGG AGAGACCGGC GCACAGAGGA
AGAGAATCTC CGCAAGAAAG GGGAGCCTCA CCACGAGCTG
CCCCCAGGGA GCACTAAGCG AGCACTGTCC AACAACACCA
GCTCCTCTCC CCAGCCAAAG AAGAAACCAC TGGATGGAGA
ATATTTCACC CTTCAGATCC GTGGGCGTGA GCGCTTCGAG
ATGTTCCGAG AGCTGAATGA GGCCTTGGAA CTCAAGGATG
CCCAGGCTGG GAAGGAGCCA GGGGGGAGCA GGGCTCACTC
CAGCCACCTG AAGTCCAAAA AGGGTCAGTC TACCTCCCGC
CATAAAAAAC TCATGTTCAA GACAGAAGGG CCTGACTCAG
ACTGACATTC TCCACTTCTT GTTCCCCACT GACAGCCTCC
CACCCCCATC TCTCCCTCCC CTGCCATTTT GGGTTTTGGG
TCTTTGAACC CTTGCTTGCA ATAGGTGTGC GTCAGAAGCA
CCCAGGACTT CCATTTGCTT TGTCCCGGGG CTCCACTGAA
CAAGTTGGCC TGCACTGGTG TTTTGTTGTG GGGAGGAGGA
TGGGGAGTAG GACATACCAG CTTAGATTTT AAGGTTTTTA
CTGTGAGGGA TGTTTGGGAG ATGTAAGAAA TGTTCTTGCA
GTTAAGGGTT AGTTTACAAT CAGCCACATT CTAGGTAGGG
GCCCACTTCA CCGTACTAAC CAGGGAAGCT GTCCCTCACT
GTTGAATTTT CTCTAACTTC AAGGCCCATA TCTGTGAAAT
GCTGGCATTT GCACCTACCT CACAGAGTGC ATTGTGAGGG
TTAATGAAAT AATGTACATC TGGCCTTGAA ACCACCTTTT
ATTACATGGG GTCTAGAACT TGACCCCCTT GAGGGTGCTT
GTTCCCTCTC CCTGTTGGTC GGTGGGTTGG TAGTTTCTAC
AGTTGGGCAG CTGGTTAGGT AGAGGGAGTT GTCAAGTCTC
TGCTGGCCCA GCCAAACCCT GTCTGACAAC CTCTTGGTGA
ACCTTAGTAC CTAAAAGGAA ATCTCACCCC ATCCCACACC
CTGGAGGATT TCATCTCTTG TATATGATGA TCTGGATCCA
CCAAGACTTG TTTTATGCTC AGGGTCAATT TCTTTTTTCT
TTTTTTTTTT TTTTTTCTTT TTCTTTGAGA CTGGGTCTCG
CTTTGTTGCC CAGGCTGGAG TGGAGTGGCG TGATCTTGGC
TTACTGCAGC CTTTGCCTCC CCGGCTCGAG CAGTCCTGCC
TCAGCCTCCG GAGTAGCTGG GACCACAGGT TCATGCCACC
ATGGCCAGCC AACTTTTGCA TGTTTTGTAG AGATGGGGTC
TCACAGTGTT GCCCAGGCTG GTCTCAAACT CCTGGGCTCA
GGCGATCCAC CTGTCTCAGC CTCCCAGAGT GCTGGGATTA
CAATTGTGAG CCACCACGTC CAGCTGGAAG GGTCAACATC
TTTTACATTC TGCAAGCACA TCTGCATTTT CACCCCACCC
TTCCCCTCCT TCTCCCTTTT TATATCCCAT TTTTATATCG
ATCTCTTATT TTACAATAAA ACTTTGCTGC CAAAAAAAAA
AAAAAAAAAA A

An amino acid sequence for human p53 is publically available in the UniProt database under accession number P04637 (SEQ ID NO: 2) and is as follows:

MEEPQSDPSVEPPLSQETFSDLWKLLPENNVLSPLPSQAMDDLMLSPD
DIEQWFTEDPGPDEAPRMPEAAPPVAPAPAAPTPAAPAPAPSWPLSSS
VPSQKTYQGSYGFRLGFLHSGTAKSVTCTYSPALNKMFCQLAKTCPVQ
LWVDSTPPPGTRVRAMAIYKQSQHMTEVVRRCPHHERCSDSDGLAPPQ
HLIRVEGNLRVEYLDDRNTFRHSVVVPYEPPEVGSDCTTIHYNYMCNS
SCMGGMNRRPILTIITLEDSSGNLLGRNSFEVRVCACPGRDRRTEEEN
LRKKGEPHHELPPGSTKRALPNNTSSSPQPKKKPLDGEYFTLQIRGRE
RFEMFRELNEALELKDAQAGKEPGGSRAHSSHLKSKKGQSTSRHKKLM
FKTEGPDSD

Without wishing to be bound by any scientific theory, positions 1-320 of the amino acid sequence shown above may correspond to or be part of a Cell Cycle And Apoptosis Regulator 2 (CCAR2) interacting region, positions 1-83 may correspond to or be part of a (HnRNP Methyltransferase, S. Cerevisiae)-Like 2 (HRMT1L2) interacting region, positions 1-44 may correspond to a transcription activation region, positions 66-110 may correspond to or be part of a WW Domain-Containing Oxidoreductase (WWOX) interacting region, positions 100-370 may correspond to or be part of a Homeodomain Interacting Protein Kinase 1 (HIPK1) interacting region, positions 100-300 may be required for interaction with Zinc Finger Protein 385A (ZNF385A), positions 113-236 may be required for interaction with F-Box Protein 42 (FBXO42), positions 116-292 may correspond to or be part of an Axis Inhibition Protein 1 (AXIN1) interacting region, positions 241-248 may correspond to or be part of 53BP2 SH3 domain interacting region, positions 256-294 may correspond to or be part of an E4F Transcription Factor 1 (E4F1) interacting region, positions 273-280 may correspond to or be part of a DNA interacting region, positions 300-393 may correspond to or be part of a Coactivator Associated Arginine Methyltransferase 1 (CARM1) interacting region, positions 319-360 may correspond to or be part of a Homeodomain Interacting Protein Kinase 2 (HIPK2) interacting region, positions may correspond to or be part of a region involved with oligomerization, positions 359-363 may correspond to or be part of an Ubiquitin-specific-processing protease 7 (USP7) interacting region, positions 368-387 may correspond to or be part of a region that reduces or represses DNA binding.

### Detection of Protein Expression Products

Proteins such as those encoded by the p53 gene are encoded by nucleic acids. For a description of the basic paradigm of molecular biology, including the expression (transcription and/or translation) of DNA into RNA into protein, see, Alberts et al. (2002) Molecular Biology of the Cell, 4^{th} Edition Taylor and Francis, Inc., ISBN: 0815332181; and Lodish et al. (1999) Molecular Cell Biology, 4^{th} Edition W H Freeman & Co, ISBN: 071673706X. Accordingly, p53 protein can be detected, e.g., by detecting protein, or by detecting a p53 gene product.

A variety of protein detection methods are known and can be used to distinguish p53 levels. In addition to the various references noted supra, a variety of protein manipulation and detection methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein. Additional details regarding protein purification and detection methods can be found in Satinder Ahuja ed., Handbook of Bioseparations, Academic Press (2000).

Non-limiting examples of proteomic detection methods are discussed below. These can include, e.g., the use of antibodies (immunoassays), various multidimensional electrophoresis methods (e.g., 2-d gel electrophoresis), mass spectrometry based methods (e.g., SELDI, MALDI, electrospray, etc.), or surface plasmon resonance methods. For example, in MALDI, a sample may be mixed with an appropriate matrix, placed on the surface of a probe and examined by laser desorption/ionization. The technique of MALDI is well known in the art. See, e.g., U.S. Pat. No. 5,045,694 (Beavis et al.), U.S. Pat. No. 5,202,561 (Gleissmann et al.), and U.S. Pat. No. 6,111,251 (Hillenkamp) Similarly, for SELDI, a first aliquot may be contacted with a solid support-bound (e.g., substrate-bound) adsorbent. A substrate is typically a probe (e.g., a biochip) that can be positioned in an interrogatable relationship with a gas phase ion spectrometer. SELDI is also a well-known technique, and has been applied to diagnostic proteomics. See, e.g. Issaq et al. (2003) “SELDI-TOF MS for Diagnostic Proteomics” Analytical Chemistry 75:149A-155A.

Antibodies to particular proteins or to their modified forms may be used to detect protein levels. Non-limiting examples include enzyme-linked immunosorbent assay (ELISA), radioimmunoassays, fluoroimmunoassays, Western blots, and immunohistochemistry (IHC). ELISA can be used to detect and quantitatively measure proteins in samples. The Western blot can be used for detection and quantification of individual proteins, where in an initial step a complex protein mixture is separated using, e.g., SDS-PAGE and then the protein of interested is identified using an antibody.

### Binding Ligands for Biomarkers

As used herein, the term “antibody” refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. By “specifically bind” or “immunoreacts with” is meant that the antibody reacts with one or more antigenic determinants of the desired antigen and does not react (i.e., bind) with other polypeptides or binds at much lower affinity (K_{d}>10⁻⁶) with other polypeptides.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ea., 2nd ed. Raven Press, N.Y. (1989)). The variable regions of each light/heavy chain pair form the antibody binding site.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs.” Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

As used herein, the term “epitope” includes any protein determinant capable of specific binding to an immunoglobulin, an scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≤1 μM; preferably ≤100 nM and most preferably ≤10 nM.

Antibodies can be produced according to any method known in the art.

Methods of preparing monoclonal antibodies are known in the art. For example, monoclonal antibodies may be prepared using hybridoma methods, such as those described by Kohler and Milstein (1975) Nature 256:495. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a full length protein or a fragment thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (see pp. 59-103 in Goding (1986) Monoclonal Antibodies: Principles and Practice Academic Press). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In some examples the antibodies to an epitope for an interested protein as described herein or a fragment thereof are humanized antibodies. Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al. 1986. Nature 321:522-525; Riechmann et al. 1988. Nature 332:323-329; Presta. 1992. Curr. Op. Struct. Biol. 2:593-596). Humanization can be essentially performed following methods of Winter and co-workers (see, e.g., Jones et al. 1986. Nature 321:522-525; Riechmann et al. 1988. Nature 332:323-327; and Verhoeyen et al. 1988. Science 239:1534-1536), by substituting rodent CDRs or CDR sequences for the corresponding sequences of a human antibody. Accordingly, such humanized antibodies are chimeric antibodies (e.g., U.S. Pat. No. 4,816,567), wherein substantially less than an intact human variable domain has been substituted by the corresponding sequence from a non-human species.

In some examples the antibodies to an epitope of an interested protein as described herein or a fragment thereof are human antibodies. Human antibodies can also be produced using various techniques known in the art, including phage display libraries (Hoogenboom and Winter. 1991. J. Mol. Biol. 227:381-388; Marks et al. 1991. J. Mol. Biol. 222:581-597) or the preparation of human monoclonal antibodies (e.g., Cole et al. 1985. Monoclonal Antibodies and Cancer Therapy Liss; Boerner et al. 1991. J. Immunol. 147(1):86-95). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in most respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al. 1992. Bio/Technology 10:779-783; Lonberg et al. 1994. Nature 368:856-859; Morrison. 1994. Nature 368:812-13; Fishwild et al. 1996. Nature Biotechnology 14:845-51; Neuberger. 1996. Nature Biotechnology 14:826; Lonberg and Huszar. 1995. Intern. Rev. Immunol. 13:65-93. U.S. Pat. No. 6,719,971 also provides guidance to methods of generating humanized antibodies.

Exemplary antibodies against p53 protein include, but are not limited to, antibodies obtained from ThermoFisher Scientific (Cambridge, Mass., USA) (e.g., Cat. No. MA5-12557, MA5-12571, MA5-14067, MA5-12453, MA5-14516, MA5-14467, MA5-12554, MA1-19055, MA1-12648 and others) and antibodies obtained from Santa Cruz Biotechnology, Inc. (Dallas, Tex., USA) (e.g., sc-126, sc-98, sc-136023, sc-71817, sc-56182, sc-56180, sc-81168, sc-71821 and others).

A non-limiting example of an anti-p53 antibody is DO-7 (Ventana Medical Systems, Tucson, Ariz., USA). DO-7 is a mouse monoclonal IgG_{2b} antibody that recognizes an epitope mapping between amino acids 1-45 (e.g., amino acids 20-25) of human p53. DO-7 is useful for detecting of both wild-type and mutant p53 protein under denaturing and non-denaturing conditions of human origin by, e.g., Western blot (WB), immunoprecipitation (IP), Immunofluorescence Microscopy (IF), IHC (paraffin) [IHC(P)] and ELISA.

### Amplification-Based Detection Methods

PCR and RT-PCR are non-limiting examples of amplification and amplification-detection methods for amplifying nucleic acids of interest (e.g., those that encode a protein of interest), facilitating detection of the nucleic acids of interest. Details regarding the use of these and other amplification methods are known in the art. Many available biology texts also have extended discussions regarding PCR and related amplification methods. One of skill will appreciate that essentially any RNA can be converted into a double stranded DNA suitable for restriction digestion, PCR expansion and sequencing using reverse transcriptase and a polymerase (“Reverse Transcription-PCR”, or “RT-PCR”). These methods can also be used to quantitatively amplify mRNA or corresponding cDNA, providing an indication of expression levels of mRNA that correspond to a gene product corresponding to p53 in an individual.

### Real Time Amplification/Detection Methods

In one aspect, real time PCR is performed on the amplification mixtures described herein, e.g., using molecular beacons or TaqMan™ probes. A molecular beacon (MB) is an oligonucleotide or peptide nucleic acid (PNA) which, under appropriate hybridization conditions, self-hybridizes to form a stem and loop structure. The MB has a label and a quencher at the termini of the oligonucleotide or PNA; thus, under conditions that permit intra-molecular hybridization, the label is typically quenched (or at least altered in its fluorescence) by the quencher. Under conditions where the MB does not display intra-molecular hybridization (e.g., when bound to a target nucleic acid, e.g., to a region of an amplicon during amplification), the MB label is unquenched. Details regarding standard methods of making and using MBs are well established in the literature and MBs are available from a number of commercial reagent sources. See also, e.g., Leone et al. (1995) “Molecular beacon probes combined with amplification by NASBA enable homogenous real-time detection of RNA.” Nucleic Acids Res. 26:2150-2155; Tyagi and Kramer (1996) “Molecular beacons: probes that fluoresce upon hybridization” Nature Biotechnology 14:303-308; Blok and Kramer (1997) “Amplifiable hybridization probes containing a molecular switch” Mol Cell Probes 11:187-194; Hsuih et al. (1997) “Novel, ligation-dependent PCR assay for detection of hepatitis C in serum” J Clin Microbiol 34:501-507; Kostrikis et al. (1998) “Molecular beacons: spectral genotyping of human alleles” Science 279:1228-1229; Sokol et al. (1998) “Real time detection of DNA:RNA hybridization in living cells” Proc. Natl. Acad. Sci. U.S.A. 95:11538-11543; Tyagi et al. (1998) “Multicolor molecular beacons for allele discrimination” Nature Biotechnology 16:49-53; Bonnet et al. (1999) “Thermodynamic basis of the chemical specificity of structured DNA probes” Proc. Natl. Acad. Sci. U.S.A. 96:6171-6176; Fang et al. (1999) “Designing a novel molecular beacon for surface-immobilized DNA hybridization studies” J. Am. Chem. Soc. 121:2921-2922; Marras et al. (1999) “Multiplex detection of single-nucleotide variation using molecular beacons” Genet. Anal. Biomol. Eng. 14:151-156; and Vet et al. (1999) “Multiplex detection of four pathogenic retroviruses using molecular beacons” Proc. Natl. Acad. Sci. U.S.A. 96:6394-6399. Additional details regarding MB construction and use is found in the patent literature, e.g., U.S. Pat. No. 5,925,517 (Jul. 20, 1999) to Tyagi et al. entitled “Detectably labeled dual conformation oligonucleotide probes, assays and kits;” U.S. Pat. No. 6,150,097 to Tyagi et al (Nov. 21, 2000) entitled “Nucleic acid detection probes having non-FRET fluorescence quenching and kits and assays including such probes” and U.S. Pat. No. 6,037,130 to Tyagi et al (Mar. 14, 2000), entitled “Wavelength-shifting probes and primers and their use in assays and kits.”

PCR detection and quantification using dual-labeled fluorogenic oligonucleotide probes, commonly referred to as “TaqMan™” probes, can also be performed according to the present subject matter. These probes are composed of short (e.g., 20-25 base) oligodeoxynucleotides that are labeled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. Accordingly, TaqMan™ probes are oligonucleotides that have a label and a quencher, where the label is released during amplification by the exonuclease action of the polymerase used in amplification. This provides a real time measure of amplification during synthesis. A variety of TaqMan™ reagents are commercially available, e.g., from Applied Biosystems (Division Headquarters in Foster City, Calif.) as well as from a variety of specialty vendors such as Biosearch Technologies (e.g., black hole quencher probes). Further details regarding dual-label probe strategies can be found, e.g., in WO92/02638.

Other similar methods include e.g. fluorescence resonance energy transfer between two adjacently hybridized probes, e.g., using the “LightCycler®” format described in U.S. Pat. No. 6,174,670.

### Amplification Primers for Marker Detection

In some embodiments, a level of p53-encoding mRNA is detected using a suitable PCR-based detection method. Aspects of the present disclosure relate to pairs of primers that are each complementary to p53-encoding mRNA. Suitable primers to be used can be designed using any suitable method. It is not intended that the present disclosure be limited to any particular primer or primer pair. For example, primers can be designed using any suitable software program, such as LASERGENE®, e.g., taking account of publicly available sequence information. The sequence of any amplicon can be detected as has already been discussed above, e.g., by hybridization, array hybridization, PCR, real-time PCR, or the like.

In some embodiments, the primers are radiolabeled, or labeled by any suitable means (e.g., using a non-radioactive fluorescent tag), to allow for rapid detection without any additional labeling step. In some embodiments, the primers are not labeled, and the amplicons are visualized following their size resolution, e.g., following agarose or acrylamide gel electrophoresis. In some embodiments, ethidium bromide staining of the PCR amplicons following size resolution allows visualization of the amplicons.

It is not intended that the primers be limited to generating an amplicon of any particular size. For example, the primers used to p53-encoding mRNA are not limited to amplifying the entire transcript or any particular subregion thereof. The primers can generate an amplicon of any suitable length for detection. In some embodiments, marker amplification produces an amplicon at least 20 nucleotides in length, or alternatively, at least 50 nucleotides in length, or alternatively, at least 100 nucleotides in length, or alternatively, at least 200 nucleotides in length.

### Computer Systems

Conventional data networking, application development and other functional aspects of the systems (and components of the individual operating components of the systems) may not be described in detail herein but are part of the invention.

The various system components discussed herein may include one or more of the following: a host server or other computing systems including a processor for processing digital data; a memory coupled to the processor for storing digital data; an input digitizer coupled to the processor for inputting digital data; an application program stored in the memory and accessible by the processor for directing processing of digital data by the processor; a display device coupled to the processor and memory for displaying information derived from digital data processed by the processor; and a plurality of databases. Various databases used herein may include: patient data such as family history, demography and environmental data, biological sample data, prior treatment and protocol data, patient clinical data, molecular profiling data of biological samples, data on therapeutic drug agents and/or investigative drugs, a gene library, a disease library, a drug library, patient tracking data, file management data, financial management data, billing data and/or like data useful in the operation of the system. As those skilled in the art will appreciate, user computer may include an operating system (e.g., Windows NT, 95/98/2000/8/10, OS2, UNIX, Linux, Solaris, MacOS, etc.) as well as various conventional support software and drivers typically associated with computers. The computer may include any suitable personal computer, network computer, workstation, minicomputer, mainframe or the like. User computer can be in a home or medical/business environment with access to a network. In an exemplary embodiment, access is through a network or the Internet through a commercially-available web-browser software package.

As used herein, the term “network” shall include any electronic communications means which incorporates both hardware and software components of such. Communication among the parties may be accomplished through any suitable communication channels, such as, for example, a telephone network, an extranet, an intranet, Internet, point of interaction device, personal digital assistant (e.g., Palm Pilot®, Blackberry®), cellular phone, kiosk, etc.), online communications, satellite communications, off-line communications, wireless communications, transponder communications, local area network (LAN), wide area network (WAN), networked or linked devices, keyboard, mouse and/or any suitable communication or data input modality. Moreover, although the system is frequently described herein as being implemented with TCP/IP communications protocols, the system may also be implemented using IPX, Appletalk, IP-6, NetBIOS, OSI or any number of existing or future protocols. If the network is in the nature of a public network, such as the Internet, it may be advantageous to presume the network to be insecure and open to eavesdroppers. Specific information related to the protocols, standards, and application software utilized in connection with the Internet is generally known to those skilled in the art and, as such, need not be detailed herein. See, for example, DILIP NAIK, INTERNET STANDARDS AND PROTOCOLS (1998); JAVA 2 COMPLETE, various authors, (Sybex 1999); DEBORAH RAY AND ERIC RAY, MASTERING HTML 4.0 (1997); and LOSHIN, TCP/IP CLEARLY EXPLAINED (1997) and DAVID GOURLEY AND BRIAN TOTTY, HTTP, THE DEFINITIVE GUIDE (2002), the contents of which are hereby incorporated by reference.

The various system components may be independently, separately or collectively suitably coupled to the network via data links which includes, for example, a connection to an Internet Service Provider (ISP) over the local loop as is typically used in connection with standard modem communication, cable modem, Dish networks, ISDN, Digital Subscriber Line (DSL), or various wireless communication methods, see, e.g., GILBERT HELD, UNDERSTANDING DATA COMMUNICATIONS (1996), which is hereby incorporated by reference. It is noted that the network may be implemented as other types of networks, such as an interactive television (ITV) network. Moreover, the system contemplates the use, sale or distribution of any goods, services or information over any network having similar functionality described herein.

As used herein, “transmit” may include sending electronic data from one system component to another over a network connection. Additionally, as used herein, “data” may include encompassing information such as commands, queries, files, data for storage, and the like in digital or any other form.

The system contemplates uses in association with web services, utility computing, pervasive and individualized computing, security and identity solutions, autonomic computing, commodity computing, mobility and wireless solutions, open source, biometrics, grid computing and/or mesh computing.

Any databases discussed herein may include relational, hierarchical, graphical, or object-oriented structure and/or any other database configurations. Common database products that may be used to implement the databases include DB2 by IBM (White Plains, N.Y.), various database products available from Oracle Corporation (Redwood Shores, Calif.), Microsoft Access or Microsoft SQL Server by Microsoft Corporation (Redmond, Wash.), or any other suitable database product. Moreover, the databases may be organized in any suitable manner, for example, as data tables or lookup tables. Each record may be a single file, a series of files, a linked series of data fields or any other data structure. Association of certain data may be accomplished through any desired data association technique such as those known or practiced in the art. For example, the association may be accomplished either manually or automatically. Automatic association techniques may include, for example, a database search, a database merge, GREP, AGREP, SQL, using a key field in the tables to speed searches, sequential searches through all the tables and files, sorting records in the file according to a known order to simplify lookup, and/or the like. The association step may be accomplished by a database merge function, for example, using a “key field” in pre-selected databases or data sectors.

More particularly, a “key field” partitions the database according to the high-level class of objects defined by the key field. For example, certain types of data may be designated as a key field in a plurality of related data tables and the data tables may then be linked on the basis of the type of data in the key field. The data corresponding to the key field in each of the linked data tables is preferably the same or of the same type. However, data tables having similar, though not identical, data in the key fields may also be linked by using AGREP, for example. In accordance with one embodiment, any suitable data storage technique may be utilized to store data without a standard format. Data sets may be stored using any suitable technique, including, for example, storing individual files using an ISO/IEC 7816-4 file structure; implementing a domain whereby a dedicated file is selected that exposes one or more elementary files containing one or more data sets; using data sets stored in individual files using a hierarchical filing system; data sets stored as records in a single file (including compression, SQL accessible, hashed via one or more keys, numeric, alphabetical by first tuple, etc.); Binary Large Object (BLOB); stored as ungrouped data elements encoded using ISO/IEC 7816-6 data elements; stored as ungrouped data elements encoded using ISO/IEC Abstract Syntax Notation (ASN.1) as in ISO/IEC 8824 and 8825; and/or other proprietary techniques that may include fractal compression methods, image compression methods, etc.

In one exemplary embodiment, the ability to store a wide variety of information in different formats is facilitated by storing the information as a BLOB. Thus, any binary information can be stored in a storage space associated with a data set. The BLOB method may store data sets as ungrouped data elements formatted as a block of binary via a fixed memory offset using either fixed storage allocation, circular queue techniques, or best practices with respect to memory management (e.g., paged memory, least recently used, etc.). By using BLOB methods, the ability to store various data sets that have different formats facilitates the storage of data by multiple and unrelated owners of the data sets. For example, a first data set which may be stored may be provided by a first party, a second data set which may be stored may be provided by an unrelated second party, and yet a third data set which may be stored, may be provided by a third party unrelated to the first and second party. Each of these three exemplary data sets may contain different information that is stored using different data storage formats and/or techniques. Further, each data set may contain subsets of data that also may be distinct from other subsets.

As stated above, in various embodiments, the data can be stored without regard to a common format. However, in one exemplary embodiment, the data set (e.g., BLOB) may be annotated in a standard manner when provided for manipulating the data. The annotation may comprise a short header, trailer, or other appropriate indicator related to each data set that is configured to convey information useful in managing the various data sets. For example, the annotation may be called a “condition header”, “header”, “trailer”, or “status”, herein, and may comprise an indication of the status of the data set or may include an identifier correlated to a specific issuer or owner of the data. Subsequent bytes of data may be used to indicate for example, the identity of the issuer or owner of the data, user, transaction/membership account identifier or the like. Each of these condition annotations are further discussed herein.

The data set annotation may also be used for other types of status information as well as various other purposes. For example, the data set annotation may include security information establishing access levels. The access levels may, for example, be configured to permit only certain individuals, levels of employees, companies, or other entities to access data sets, or to permit access to specific data sets based on the transaction, issuer or owner of data, user or the like. Furthermore, the security information may restrict/permit only certain actions such as accessing, modifying, and/or deleting data sets. In one example, the data set annotation indicates that only the data set owner or the user are permitted to delete a data set, various identified users may be permitted to access the data set for reading, and others are altogether excluded from accessing the data set. However, other access restriction parameters may also be used allowing various entities to access a data set with various permission levels as appropriate. The data, including the header or trailer may be received by a stand alone interaction device configured to add, delete, modify, or augment the data in accordance with the header or trailer.

One skilled in the art will also appreciate that, for security reasons, any databases, systems, devices, servers or other components of the system may consist of any combination thereof at a single location or at multiple locations, wherein each database or system includes any of various suitable security features, such as firewalls, access codes, encryption, decryption, compression, decompression, and/or the like.

The computing unit of the web client may be further equipped with an Internet browser connected to the Internet or an intranet using standard dial-up, cable, DSL or any other Internet protocol known in the art. Transactions originating at a web client may pass through a firewall in order to prevent unauthorized access from users of other networks. Further, additional firewalls may be deployed between the varying components of CMS to further enhance security.

Firewall may include any hardware and/or software suitably configured to protect CMS components and/or enterprise computing resources from users of other networks. Further, a firewall may be configured to limit or restrict access to various systems and components behind the firewall for web clients connecting through a web server. Firewall may reside in varying configurations including Stateful Inspection, Proxy based and Packet Filtering among others. Firewall may be integrated within an web server or any other CMS components or may further reside as a separate entity.

The computers discussed herein may provide a suitable website or other Internet-based graphical user interface which is accessible by users. In one embodiment, the Microsoft Internet Information Server (IIS), Microsoft Transaction Server (MTS), and Microsoft SQL Server, are used in conjunction with the Microsoft operating system, Microsoft NT web server software, a Microsoft SQL Server database system, and a Microsoft Commerce Server. Additionally, components such as Access or Microsoft SQL Server, Oracle, Sybase, Informix MySQL, Interbase, etc., may be used to provide an Active Data Object (ADO) compliant database management system.

Any of the communications, inputs, storage, databases or displays discussed herein may be facilitated through a website having web pages. The term “web page” as it is used herein is not meant to limit the type of documents and applications that might be used to interact with the user. For example, a typical website might include, in addition to standard HTML documents, various forms, Java applets, JavaScript, active server pages (ASP), common gateway interface scripts (CGI), extensible markup language (XML), dynamic HTML, cascading style sheets (CSS), helper applications, plug-ins, and the like. A server may include a web service that receives a request from a web server, the request including a URL (yahoo.com/stockquotes/ge) and an IP address (123.56.789.234). The web server retrieves the appropriate web pages and sends the data or applications for the web pages to the IP address. Web services are applications that are capable of interacting with other applications over a communications means, such as the internet. Web services are typically based on standards or protocols such as XML, XSLT, SOAP, WSDL and UDDI. Web services methods are well known in the art, and are covered in many standard texts. See, e.g., ALEX NGHIEM, IT WEB SERVICES: A ROADMAP FOR THE ENTERPRISE (2003), hereby incorporated by reference.

The web-based clinical database for the system and method of the present invention preferably has the ability to upload and store clinical data files in native formats and is searchable on any clinical parameter. The database is also scalable and may utilize an EAV data model (metadata) to enter clinical annotations from any study for easy integration with other studies. In addition, the web-based clinical database is flexible and may be XML and XSLT enabled to be able to add user customized questions dynamically. Further, the database includes exportability to CDISC ODM.

Practitioners will also appreciate that there are a number of methods for displaying data within a browser-based document. Data may be represented as standard text or within a fixed list, scrollable list, drop-down list, editable text field, fixed text field, pop-up window, and the like. Likewise, there are a number of methods available for modifying data in a web page such as, for example, free text entry using a keyboard, selection of menu items, check boxes, option boxes, and the like.

The system and method may be described herein in terms of functional block components, screen shots, optional selections and various processing steps. It should be appreciated that such functional blocks may be realized by any number of hardware and/or software components configured to perform the specified functions. For example, the system may employ various integrated circuit components, e.g., memory elements, processing elements, logic elements, look-up tables, and the like, which may carry out a variety of functions under the control of one or more microprocessors or other control devices. Similarly, the software elements of the system may be implemented with any programming or scripting language such as C, C++, Macromedia Cold Fusion, Microsoft Active Server Pages, Java, COBOL, assembler, PERL, Visual Basic, SQL Stored Procedures, extensible markup language (XML), with the various algorithms being implemented with any combination of data structures, objects, processes, routines or other programming elements. Further, it should be noted that the system may employ any number of conventional techniques for data transmission, signaling, data processing, network control, and the like. Still further, the system could be used to detect or prevent security issues with a client-side scripting language, such as JavaScript, VBScript or the like. For a basic introduction of cryptography and network security, see any of the following references: (1) “Applied Cryptography: Protocols, Algorithms, And Source Code In C,” by Bruce Schneier, published by John Wiley & Sons (second edition, 1995); (2) “Java Cryptography” by Jonathan Knudson, published by O'Reilly & Associates (1998); (3) “Cryptography & Network Security: Principles & Practice” by William Stallings, published by Prentice Hall; all of which are hereby incorporated by reference.

As used herein, the term “end user”, “consumer”, “customer”, “client”, “treating physician”, “hospital”, or “business” may be used interchangeably with each other, and each shall mean any person, entity, machine, hardware, software or business. Each participant is equipped with a computing device in order to interact with the system and facilitate online data access and data input. The customer has a computing unit in the form of a personal computer, although other types of computing units may be used including laptops, notebooks, hand held computers, set-top boxes, cellular telephones, touch-tone telephones and the like. The owner/operator of the system and method of the present invention has a computing unit implemented in the form of a computer-server, although other implementations are contemplated by the system including a computing center shown as a main frame computer, a mini-computer, a PC server, a network of computers located in the same of different geographic locations, or the like. Moreover, the system contemplates the use, sale or distribution of any goods, services or information over any network having similar functionality described herein.

In one exemplary embodiment, each client customer may be issued an “account” or “account number”. As used herein, the account or account number may include any device, code, number, letter, symbol, digital certificate, smart chip, digital signal, analog signal, biometric or other identifier/indicia suitably configured to allow the consumer to access, interact with or communicate with the system (e.g., one or more of an authorization/access code, personal identification number (PIN), Internet code, other identification code, and/or the like). The account number may optionally be located on or associated with a charge card, credit card, debit card, prepaid card, embossed card, smart card, magnetic stripe card, bar code card, transponder, radio frequency card or an associated account. The system may include or interface with any of the foregoing cards or devices, or a fob having a transponder and RFID reader in RF communication with the fob. Although the system may include a fob embodiment, the invention is not to be so limited. Indeed, system may include any device having a transponder which is configured to communicate with RFID reader via RF communication. Typical devices may include, for example, a key ring, tag, card, cell phone, wristwatch or any such form capable of being presented for interrogation. Moreover, the system, computing unit or device discussed herein may include a “pervasive computing device,” which may include a traditionally non-computerized device that is embedded with a computing unit. The account number may be distributed and stored in any form of plastic, electronic, magnetic, radio frequency, wireless, audio and/or optical device capable of transmitting or downloading data from itself to a second device.

As will be appreciated by one of ordinary skill in the art, the system may be embodied as a customization of an existing system, an add-on product, upgraded software, a stand alone system, a distributed system, a method, a data processing system, a device for data processing, and/or a computer program product. Accordingly, the system may take the form of an entirely software embodiment, an entirely hardware embodiment, or an embodiment combining aspects of both software and hardware. Furthermore, the system may take the form of a computer program product on a computer-readable storage medium having computer-readable program code means embodied in the storage medium. Any suitable computer-readable storage medium may be utilized, including hard disks, CD-ROM, optical storage devices, magnetic storage devices, and/or the like.

The system and method is described herein with reference to screen shots, block diagrams and flowchart illustrations of methods, apparatus (e.g., systems), and computer program products according to various embodiments. It will be understood that each functional block of the block diagrams and the flowchart illustrations, and combinations of functional blocks in the block diagrams and flowchart illustrations, respectively, can be implemented by computer program instructions.

The steps recited in any of the method or process descriptions herein may be executed in any order and are not limited to the order presented.

Computer program instructions may be loaded onto a general purpose computer, special purpose computer, or other programmable data processing apparatus to produce a machine, such that the instructions that execute on the computer or other programmable data processing apparatus create means for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

Benefits, other advantages, and solutions to problems have been described herein with regard to specific embodiments. However, the benefits, advantages, solutions to problems, and any element(s) that may cause any benefit, advantage, or solution to occur or become more pronounced are not to be construed as critical, required, or essential features or elements of any or all the claims or the invention. The scope of the invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described exemplary embodiments that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims.

No claim element herein is to be construed under the provisions of 35 U.S.C. § 112, sixth paragraph, unless the element is expressly recited using the phrase “means for.”

### General Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the term “about” in the context of a numerical value or range means ±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

A small molecule is a compound that is less than 2000 daltons in mass. The molecular mass of the small molecule is preferably less than 1000 daltons, more preferably less than 600 daltons, e.g., the compound is less than 500 daltons, 400 daltons, 300 daltons, 200 daltons, or 100 daltons.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. Purified also defines a degree of sterility that is safe for administration to a human subject, e.g., lacking infectious or toxic agents. In the case of tumor antigens or markers (e.g., p53), the antigen may be purified or a processed preparation such as a tumor cell lysate.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “subject,” “patient,” “individual,” and the like as used herein are not intended to be limiting and can be generally interchanged. That is, an individual described as a “patient” does not necessarily have a given disease, but may be merely seeking medical advice.

As used herein, the term “subject” refers to any animal (e.g., a mammal), including, but not limited to, humans, non-human primates, canines, felines, rodents, and the like. In some aspects, the subject is a mammal, and in some aspects, the subject is a human. The methods are also applicable to companion animals such as dogs and cats as well as livestock such as cows, horses, sheep, goats, pigs, and other domesticated and wild animals.

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

As used herein, “treatment” is an approach for obtaining beneficial or desired results, including clinical results. Beneficial or desired clinical results include, but are not limited to, alleviation or amelioration of symptoms, diminishment of extent of disease, stabilized (i.e., not worsening) state of disease, preventing spread of disease, delay or slowing of disease progression, amelioration or palliation of the disease state, and recovery (whether partial or total), whether detectable or undetectable. “Treatment” can also include prolonging survival as compared to expected survival if not receiving treatment. As used herein, “inhibition” of disease progression or a disease complication in a subject means preventing or reducing the disease progression and/or disease complication in the subject.

Insofar as the methods of the present disclosure are directed to compositions and methods for treating a disease or disease state, it is understood that the term “prevent” does not require that the disease state (e.g., dysplasia or esophageal cancer) be completely thwarted. The term “prevent” can encompass partial effects when the treatments disclosed herein are administered as a prophylactic measure. The prophylactic measures include, without limitation, the administration of compound, surgical intervention, or treatment regimen to an individual who is at risk of developing, e.g., dysplasia or esophageal cancer.

As used herein, a “symptom” associated with a disorder includes any clinical or laboratory manifestation associated with the disorder, and is not limited to what the subject can feel or observe.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

As used herein, “pharmaceutically acceptable” carrier or excipient refers to a carrier or excipient that is suitable for use with humans and/or animals without undue adverse side effects (such as toxicity, irritation, and allergic response) commensurate with a reasonable benefit/risk ratio. It can be, e.g., a pharmaceutically acceptable solvent, suspending agent or vehicle, for delivering the instant compounds to the subject.

An “assay” is an investigative procedure for qualitatively assessing or quantitatively measuring the presence, amount, or functional activity of a target entity (e.g., the level of p53 protein or p53-encoding mRNA in a test sample). The term “assaying” does not include the mere reading of a database entry or report.

A “control” sample or value refers to a sample that serves as a reference, usually a known reference, for comparison to a test sample. For example, a test sample can be taken from a test subject, e.g., a subject in need of prognosis or a risk determination for dysplasia and/or esophageal cancer. A control can also represent an average value gathered from a number of results. One of skill in the art will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are variable in controls, variation in test samples will not be considered as significant.

The “normal amount” of a compound (e.g., a protein such as p53 or a polynucleotide such as p53-encoding mRNA) refers to a normal amount of the compound in an individual (e.g., within a test sample obtained or provided from said individual) known not to be diagnosed with BE, dysplasia, or esophageal cancer (depending on the context). The amount of the protein can be measured in a test sample and compared to the “normal control level,” utilizing techniques such as reference limits, discrimination limits, or risk defining thresholds to define cutoff points and abnormal values (e.g., for dysplasia or esophageal cancer). The normal control level means the level of one or more compounds (e.g., a p53 protein or a p53-encoding mRNA) typically found in a subject known not to suffer from BE, dysplasia, or esophageal cancer (depending on the context). Such normal control levels and cutoff points may vary based on whether a protein is used alone or in a formula combining with other proteins into an index. Alternatively, the normal control level can be a database of protein patterns from previously tested subjects who did not convert to dysplasia or esophageal cancer over a clinically relevant time horizon.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being assessed in that the control does not suffer from the disease in question or is not at risk for the disease.

Relative to a control level, the level that is determined may an increased level. As used herein, the term “increased” with respect to level (e.g., protein level) refers to any % increase above a control level. In embodiments, the increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

Relative to a control level, the level that is determined may a decreased level. As used herein, the term “decreased” with respect to level (e.g., protein level) refers to any % decrease below a control level. In embodiments, the decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

“Risk” in the context of the present disclosure, relates to the probability that an event will occur, as in the conversion to dysplasia, and can mean a subject's “absolute” risk or “relative” risk. In embodiments, the risk relates to the probability that an event will occur over a specific time period. In various embodiments, a “high risk” subject may comprise a subject who is likely to develop esophageal cancer within, e.g., about 1, 2, 3, 4, or 5 years. In some embodiments, an “intermediate risk” subject may comprise a subject who is likely to develop esophageal cancer within, e.g., about 6, 7, 8, 9, or 10 years. In certain embodiments, a “low risk” subject may comprise a subject who is unlikely to develop esophageal cancer within, e.g., 10, 15, 20 or more years. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1: Development of p53 IHC Test Thresholds and a Case-Control Study of BE Progression

It was hypothesized that p53 mutations, and thus abnormalities in p53 expression, are present in non-dysplastic Barrett's mucosa prior to the development of dysplasia, and thus can be used to identify patients at increased risk of progression to dysplasia. If true, non-dysplastic Barrett's biopsies in patients with simultaneous high grade dysplasia should have a high frequency of abnormal p53 expression. To test this hypothesis, p53 expression was examined in 17 non-dysplastic biopsies from patients without dysplasia and 115 non-dysplastic biopsies from patients with concurrent high grade dysplasia Immunohistochemical stains for p53 were performed using the DO-7 antibody (Ventana Medical Systems, Tucson, Ariz., USA). Stains were performed on the BenchMark XT or BenchMark ULTRA automated slide staining systems with the OptiView or UltraView detection kits (Ventana Medical Systems, Tucson, Ariz., USA) according to the manufacturer's recommendations. Positive and negative controls were included in all staining runs. The percentage of nuclei with positive staining (in increments of 5 to 10%) was scored on an intensity scale of 0-3, with 0+ representing no staining and 3+ representing very strong staining.

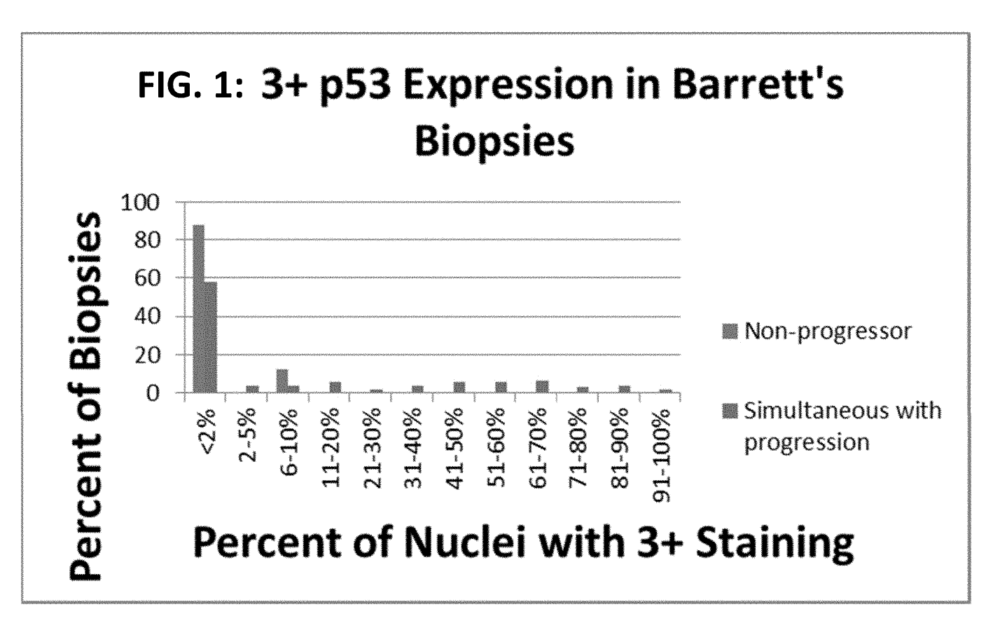

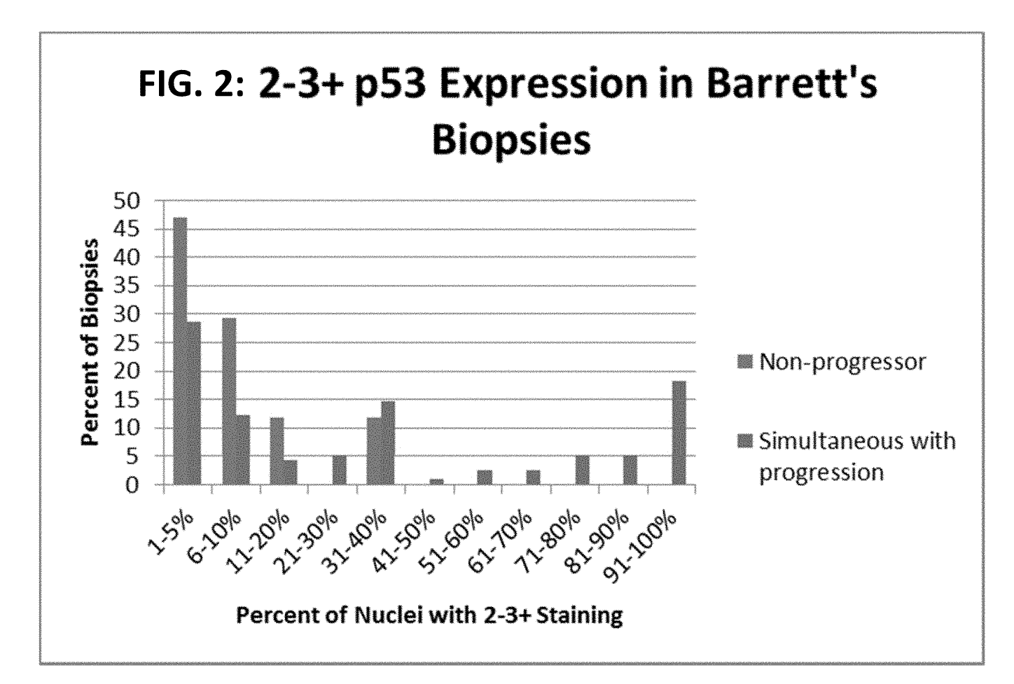

p53 expression was examined by IHC in non-dysplastic esophageal biopsies from patients without high grade dysplasia in a concurrent biopsy, and the normal p53 expression pattern was identified and defined as follows: 1. Crypt base epithelial cells had 3+ nuclear positivity in <2% of cells, 2+ nuclear positivity in ≤20% of cells, and 1+ nuclear positivity in the majority of remaining cells (FIGS. 1 and 2). 2. Surface epithelial cells generally had only 0-1+ nuclear positivity, and no 2+ or 3+ nuclear positivity, except in the setting of inflammatory injury, in which case a surface staining pattern similar to crypt base cells may be seen. This normal p53 staining pattern was found in most crypts in all biopsies from patients without dysplasia, and in many crypts from most patients (94%) with concurrent dysplasia.

p53 expression was examined by IHC in non-dysplastic biopsies from patients with concurrent high grade dysplasia to identify and define abnormal p53 expression. Two patterns of abnormal p53 expression in crypt epithelial cells were identified, that differed from the normal p53 staining described above. The first abnormal p53 expression pattern consisted of increased p53 expression (3+ nuclear positivity in >1% of cells and/or 2+ nuclear positivity in ≥20% of cells). It was found in 45% of biopsies, and involved a variable amount of the biopsy samples, from as much as all of the crypts to as little as a single crypt. Utilizing these criteria, abnormally increased p53 expression could be determined in foci as small as a single crypt. Further, abnormal expression of p53 did not always extend to involve surface epithelial cells, and could be present only in the crypt base. Two different scoring thresholds were tested, 3+ positivity and combined 2-3+ positivity. It was determined that a threshold of 2-3+ positivity in >50% of cells provided the greatest degree of discrimination between non-dysplastic biopsies with or without concurrent high grade dysplasia (34% sensitivity and 100% specificity). Biopsies with 2-3+ positivity in 20-50% of cells were considered equivocal for abnormal p53. This pattern was also associated with concurrent high grade dysplasia (21% sensitivity and 88% specificity).

The second pattern of abnormal p53 expression identified was complete loss of p53 expression in crypt epithelial nuclei (0+ staining in 100% of cells). Similar to the abnormal increased p53 expression pattern, this absent staining pattern could be seen in many crypts, or in foci as small as a single crypt. This pattern was identified in non-dysplastic biopsies from patients with concurrent high grade dysplasia but not in any of the non-dysplastic biopsies from patients without dysplasia (sensitivity 17% and specificity 100%).

To further validate the thresholds for abnormal p53 expression, fifty BE biopsies with no dysplasia and 50 BE biopsies with high grade dysplasia were identified. Immunohistochemical stains for p53 were performed using the DO-7 antibody (Ventana Medical Systems, Tucson, Ariz., USA). Stains were performed on the BenchMark XT or BenchMark ULTRA automated slide staining systems with the OptiView or UltraView detection kits (Ventana Medical Systems, Tucson, Ariz., USA) according to the manufacturer's recommendations. Positive and negative controls were included in all staining runs. Using the scoring methods and thresholds defined above, only 4% of negative cases had abnormal p53, compared to 96% of high grade dysplastic biopsies, yielding a sensitivity of 96% for high grade dysplasia and a specificity of 96%.

A case-control study of BE progression was conducted. Progressors (cases) were defined as patients with a baseline biopsy of BE negative for dysplasia, followed by a subsequent biopsy with BE and high grade dysplasia or adenocarcinoma. Non-progressors (controls) were defined as patients with a baseline biopsy of BE negative for dysplasia, followed by a subsequent biopsy at least three years later with BE negative for dysplasia. Controls were age and gender matched to cases.

192 potential progressors were identified and matched to 196 non-progressors. Following central histopathologic review, 175 progressors and 185 non-progressors remained eligible for study (defined by confirmation of histopathologic diagnosis, and availability of tissue for p53 IHC staining). All baseline biopsies were stained for p53 and evaluated using the threshold criteria described above.

Baseline biopsies in progressors were much more likely to have abnormal p53 than non-progressors. The 175 progressors had a total of 256 baseline biopsies with BE negative for dysplasia, and 97 of these (37.9%) had an abnormal p53 IHC test. The 185 non-progressors had a total of 201 baseline biopsies with BE negative for dysplasia, and 4 of these (2%) had an abnormal p53 IHC test. These results indicate that an abnormal p53 IHC test is strongly associated with progression to high grade dysplasia or adenocarcinoma (P<0.0001, Chi square test). Furthermore, many of the abnormal p53 IHC test results in progressors were present in biopsies performed more than 5 years prior to the development of high grade dysplasia or cancer.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for detecting p53 expression in an immunohistological (IHC) sample, comprising
(a) immunohistologically staining a biopsy from a subject for p53 expression, wherein said biopsy comprises cellular nuclei, thereby forming an immunohistological (IHC) sample comprising a plurality of nuclei; and
(b) identifying p53 expression as abnormal in the IHC sample if (i) a proportion of nuclei in the plurality of nuclei has an intensity of p53 protein staining of at least 2+ on a scale from 0+ to 3+, wherein a threshold proportion of at least 50% of the nuclei having an intensity of p53 protein staining of at least 2+ indicates abnormal expression, or (ii) 100% of the nuclei in the plurality of nuclei has an intensity of p53 protein staining of 0+.

2. The method of claim 1, wherein the proportion of nuclei with positive p53 staining is rounded to the nearest 5%.

3. The method of claim 1, wherein said threshold yields a sensitivity of 96% and a specificity of 96% for differentiating non-dysplastic from high grade dysplastic samples, and is strongly associated with subjects who will develop high grade dysplasia (P<0.0001).

4. A method for identifying whether a subject who does not have dysplasia is at risk of developing dysplasia or esophageal cancer, comprising
(a) providing a test sample from said subject;
(b) assaying the level of p53 protein or p53-encoding mRNA in the test sample; and
(c) identifying the subject as at risk of developing dysplasia or esophageal cancer if the level of p53 protein or p53-encoding mRNA in the test sample is elevated or reduced compared to a normal control.

5. The method of claim 4, wherein the subject is identified as at risk of developing dysplasia or esophageal cancer if the level of p53 protein or p53-encoding mRNA in the test sample is at least about 5%, 10%, 15%, 20%, 25%, 30%, 35%, 40%, 45%, 50%, 55%, 60%, 70%, 80%, 90%, 1-fold, 2-fold, 3-fold, 4-fold, 5-fold higher in said test sample compared to a normal control.

6. The method of claim 4, wherein the subject is identified as at risk of developing dysplasia or esophageal cancer if the level of p53 protein or p53-encoding mRNA in the test sample is reduced by at least about 5%, 10%, 15%, 20%, 25%, 30%, 35%, 40%, 45%, 50%, 55%, 60%, 70%, 80%, or 90% in said test sample compared to a normal control.

7. The method of claim 4, wherein the test sample comprises a biopsy.

8. The method of claim 7, wherein the biopsy comprises a tissue biopsy.

9. The method of claim 8, wherein the tissue biopsy comprises an esophagus tissue biopsy.

10. The method of claim 9, wherein the esophagus tissue biopsy comprises a Barrett's esophagus (BE) biopsy.

11. The method of claim 4, wherein the subject has BE.

12. The method of claim 11, wherein the BE comprises
(i) a circumferential extent of metaplasia that is less than about 0.25, 0.5, 0.75, 1, 1.25, 1.5, 2.0, 2.5, 3.0, 3.5, 4.0, 4.5, or 5 or at least about 0.25, 0.5, 0.75, 1, 1.25, 1.5, 2.0, 2.5, 3.0, 3.5, 4.0, 4.5, or 5 centimeters (cm); and/or
(ii) a maximum extend of metaplasia that is less than about 0.25, 0.5, 0.75, 1, 1.25, 1.5, 2.0, 2.5, 3.0, 3.5, 4.0, 4.5, or 5 cm or at least about 0.25, 0.5, 0.75, 1, 1.25, 1.5, 2.0, 2.5, 3.0, 3.5, 4.0, 4.5, or 5 cm.

13. The method of claim 4, wherein the subject does not have BE.

14. The method of claim 1, wherein the subject is afflicted with gastroesophageal reflux disease.

15. The method of claim 14, wherein the subject suffers from heartburn, chronic cough, laryngitis, or nausea.

16. The method of claim 14, wherein the subject has had gastroesophageal reflux disease for at least about 1, 2, 3, 4, or 5 years.

17. The method of claim 4, wherein the subject has hiatal hernia, is at least 50 years of age, self-identifies as white or Caucasian, and/or is overweight.

18. The method of claim 4, further comprising directing or advising the subject to obtain (i) additional screening or additional diagnostic testing for esophageal dysplasia or esophageal cancer; or (ii) treatment to reduce, delay, or prevent the onset or progression of dysplasia or esophageal cancer.

19. The method of claim 4, further comprising directing or advising the subject to (i) eat less fatty food, chocolate, caffeine, spicy food, or peppermint; (ii) avoid alcohol, caffeinated beverages, or tobacco; or (iii) lose weight.

20. The method of claim 4, further comprising administering to said subject (i) a proton pump inhibitor; (ii) an antacid; (iii) radiofrequency ablation (RFA); (iv) photodynamic therapy (PDT); (v) endoscopic spray cryotherapy; or (vi) endoscopic mucosal resection (EMR).

21. The method of claim 4, wherein the dysplasia comprises low-grade dysplasia or high-grade dysplasia.

22. The method of claim 4, wherein the test sample comprises esophageal cells, and the level of p53 protein is the level of p53 protein in the nuclei of the esophageal cells.

23. The method of claim 22, wherein the level of p53 protein in the nuclei of the esophageal cells comprises
(i) the proportion of nuclei having an amount of p53 protein that is 5%, 10%, 15%, 20%, 25%, 30%, 35%, 40%, 45%, 50%, 55%, 60%, 70%, 80%, 90%, 1-fold, 2-fold, 3-fold, 4-fold, 5-fold higher than the normal amount of p53 protein in an esophageal cell nucleus; or
(ii) the proportion of nuclei having an amount of p53 protein that is least about 5%, 10%, 15%, 20%, 25%, 30%, 35%, 40%, 45%, 50%, 55%, 60%, 70%, 80%, or 90% lower than the normal amount of p53 protein in an esophageal cell nucleus.

24. A method for monitoring the development of dysplasia or esophageal cancer in a subject who has been diagnosed with Barrett's esophagus (BE) but does not have dysplasia, comprising periodically determining the level of p53 protein or p53-encoding mRNA in said subject, and identifying dysplasia or esophageal cancer as developing if the level of p53 increases or decreases over time, wherein determining the level of p53 protein or p53-encoding mRNA comprises
(a) providing a test sample from said subject; and
(b) assaying the level of p53 protein or p53-encoding mRNA in the test sample.

25. The method of claim 24, wherein the level of p53 protein or p53-encoding mRNA is determined at least once every 1, 2, 3, 4, 6, 12, 18, or 24 months.

26. A method for determining a prognosis for a subject who has been diagnosed with Barrett's esophagus (BE) but does not have dysplasia, comprising
(a) providing a test sample from said subject;
(b) assaying the level of p53 protein or p53-encoding mRNA in the test sample; and
(c) comparing the level of p53 protein or p53-encoding mRNA to a value in a database to identify the subject's risk of suffering from dysplasia or esophageal cancer.

27. The method of claim 26, wherein said database contains (i) p53 protein or p53-encoding mRNA level values from subjects who have developed dysplasia or esophageal cancer; (ii) index values calculated based on p53 protein or p53-encoding mRNA levels in subjects who have developed dysplasia or esophageal cancer; (iii) p53 protein or p53-encoding mRNA level values from subjects who have developed dysplasia or esophageal cancer at various time points after the p53 protein or p53-encoding mRNA level values were provided from said subjects; (iv) index values calculated based on p53 protein or p53-encoding mRNA levels in subjects who have developed dysplasia or esophageal cancer at various time points after the p53 protein or p53-encoding mRNA level values were provided from said subjects; and/or (v) absolute or relative risk values calculated based on p53 protein or p53-encoding mRNA level values from subjects who have developed dysplasia or esophageal cancer.

28. The method of claim 27, wherein said absolute or relative risk values comprise mean or median level values calculated using p53 protein or p53-encoding mRNA level values from subjects who have developed dysplasia or esophageal cancer.

29. The method of claim 4, wherein assaying the level of p53 protein or p53-encoding mRNA comprises contacting p53 protein or p53-encoding mRNA in the test sample with a p53-specific binding agent.

30. The method of claim 29, wherein said binding agent comprises an antibody or a fragment thereof.

31. The method of claim 30, wherein said antibody is an anti-p53 antibody.

32. The method of claim 29, wherein said p53-specific binding agent is attached to a solid support.

33. The method of claim 4, wherein said assaying comprises an enzyme-linked immunosorbent assay (ELISA), a radioimmunoassay, a fluoroimmunoassay, a Western blot, or immunohistochemistry (IHC).

34. The method of claim 29, wherein said binding agent comprises a primer, a pair of primers, or an oligonucleotide probe.

35. The method of claim 4, wherein said assaying comprises a reverse transcriptase polymerase chain reaction (RT-PCR), quantitative PCT (qPCR), microarray analysis, or in situ hybridization.

36. A method for identifying whether a subject who does not have dysplasia is at risk of developing dysplasia or esophageal cancer, comprising
(a) immunohistologically staining a biopsy from a subject for p53 expression, wherein said biopsy comprises cellular nuclei, thereby forming an immunohistological (IHC) sample comprising a plurality of nuclei; and
(b) identifying p53 expression as abnormal in the IHC sample if (i) a proportion of nuclei in the plurality of nuclei has an intensity of p53 protein staining of at least 2+ on a scale from 0+ to 3+, wherein a threshold proportion of at least 50% of the nuclei having an intensity of p53 protein staining of at least 2+ indicates abnormal expression, or (ii) 100% of the nuclei in the plurality of nuclei has an intensity of p53 protein staining of 0+; and
(c) identifying the subject as at risk of developing dysplasia or esophageal cancer if p53 staining in said IHC sample indicates abnormal p53 expression.

37. A method of treating or monitoring a subject identified as risk of developing dysplasia or esophageal cancer according to the method of claim 36 for dysplasia or esophageal cancer, comprising obtaining an additional biopsy from said subject or administering (i) a proton pump inhibitor; (ii) an antacid; (iii) radiofrequency ablation (RFA); (iv) photodynamic therapy (PDT); (v) endoscopic spray cryotherapy; and/or (vi) endoscopic mucosal resection (EMR) to the subject.

38. A diagnostic system comprising
(a) an assortment, collection, or compilation of test results data scoring, representing, including, or corresponding to the level of p53 protein or p53-encoding mRNA in a plurality of test samples;
(b) a means for computing an index value using said level, wherein the index value comprises a diagnostic, prognostic, or progression scores; and
(c) a means for reporting the index value.

39. A kit comprising
(a) a p53-specific binding agent for detecting the level of p53 protein or p53-encoding mRNA, and
(b) instructions for using the agent for determining whether a subject is at risk of developing dysplasia or esophageal cancer, for monitoring the progression from Barrett's esophagus (BE) to dysplasia or esophageal cancer, and/or for determining the prognosis of the subject.
